(19) United States
(12) Patent Application Publication
(10) Pub. No.: US 2026/0335325 A1
(43) Pub. Date: Oct. 29, 2026

(54) PHARMACEUTICAL COMPOSITION FOR INHALATION POWDER AEROSOL AND PROCESS FOR PREPARATION THEREOF

(71) Applicant: SICHUAN HAISCO PHARMACEUTICAL CO., LTD., Chengdu (CN)

(72) Inventors: Liang LI, Chengdu (CN); Chong FENG, Chengdu (CN); Jingfei WANG, Chengdu (CN); Yongqi CUI, Chengdu (CN); Xuanmiao ZHANG, Chengdu (CN); Dong REN, Chengdu (CN); Ying DOU, Chengdu (CN); Er TAN, Chengdu (CN)

(21) Appl. No.: 19/651,081

(22) Filed: Apr. 17, 2026

(30) Foreign Application Priority Data
Apr. 17, 2025 (CN) 202510483246.7

(51) Int. Cl. A61K 31/519; A61K 9/00; A61K 9/16; A61K 9/48

(52) U.S. Cl. CPC A61K 31/519; A61K 9/0075; A61K 9/1623; A61K 9/4816; A61K 9/4833; A61K 9/4858

(57) ABSTRACT

The present invention relates to a pharmaceutical composition of the compound of Formula (I) or a stereoisomer, tautomer, or pharmaceutically acceptable salt thereof, as well as a pharmaceutical formulation thereof. The present invention also relates to a process for preparing the pharmaceutical composition and the pharmaceutical formulation, and use of the same in the manufacture of a medicament for treating a PDE3/4-mediated disease.

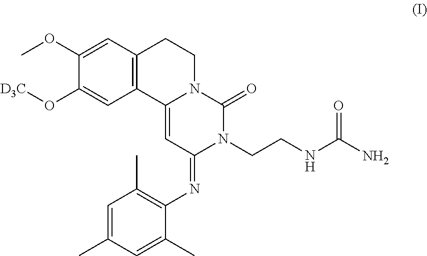 [SMILES: [2H]C([2H])([2H])Oc1cc2c(cc1OC)CCn1c-2c/c(=N\c2c(C)cc(C)cc2C)n(CCNC(N)=O)c1=O]

### CROSS REFERENCE TO RELATED APPLICATIONS

This application claims the benefit of Chinese Application No. 202510483246.7, filed Apr. 17, 2025, which is incorporated by reference herein in its entirety.

### TECHNICAL FIELD

The present invention relates to the field of chemical pharmaceutical technology, specifically, to a pharmaceutical composition for inhalation powder aerosol and process for preparation thereof.

### BACKGROUND OF THE INVENTION

Globally, there are approximately 300 million asthma patients and 250 million chronic obstructive pulmonary disease (COPD) patients. Over the past decade, with air pollution and environmental degradation worldwide, the incidence and mortality rates of asthma have shown an upward trend, with more than 180,000 deaths from asthma each year. Additionally, analyses have shown that in 2020, COPD has risen from the sixth leading cause of death to the third worldwide. Clinically therapeutic drugs for COPD are primarily categorized into long-acting β2-agonists (LABAs), long-acting anticholinergic drugs (e.g., LAMAs), and steroid hormones. The compound of Formula (I) is a PDE3/PDE4 dual inhibitor which acts on targets distinct from those of other compounds, providing an alternative treatment option when the aforementioned targets are ineffective or when a patient cannot use the aforementioned drugs.

Systemic side effects of orally administered drugs, such as AstraZeneca's selective PDE4 inhibitor roflumilast (Daxas), often limit their use in the treatment of severe COPD patients. In contrast, inhalation formulations leverage local administration, minimizing adverse reactions thereof. Inhaled PDE inhibitors offer a viable option to improve tolerability and maximize their potential for treating respiratory diseases.

An inhalation powder aerosol brings drugs into the respiratory tract by delivery with a carrier (e.g., lactose) onto the surface of which the drugs are adsorbed. Due to the limited delivery capacity of the carrier, existing inhalation powder aerosols have low drug loading capacities and delivery efficiencies; and even when the single dose is below 500 μg, the amount of effectively inhaled drug reaching the respiratory tract is often only 20-30%. Inhalation powder aerosols also contain micronized drugs, which, due to their small particle size (less than 10 μm), have a larger surface area per unit mass of the pharmaceutical powder, stronger adhesion, and higher tendency to agglomerate, resulting in poor powder flowability. During preparation, problems with non-uniform drug blending often occur. Non-uniform pharmaceutical powder can cause fluctuations in drug dosage when inhaled by a patient, posing risks of reduced therapeutic efficacy or increased toxicity and side effects of the drug. Meanwhile, inhalation powder aerosols are relatively sensitive to environmental moisture; consequently, existing technologies typically face difficulties in obtaining inhalation powder aerosols with high stability. As described in CN101829072A, obtaining inhalation powder aerosols with good stability typically necessitates the use of complex preparation processes.

The compound of Formula (I), as a dual PDE inhibitor, can provide an additional or synergistic effect, and when developed as an inhalation powder aerosol, can fully exert its therapeutic effect, reduce systemic exposure, and provide enhanced compliance and tolerability to patients.

### SUMMARY OF THE INVENTION

An object of the present invention is to provide a pharmaceutical composition or pharmaceutical formulation. The pharmaceutical composition or pharmaceutical formulation comprises a therapeutically effective amount of an active ingredient and a pharmaceutical excipient, wherein the active ingredient is selected from the compound of Formula (I), or a stereoisomer, tautomer, pharmaceutically acceptable salt thereof. The present invention also relates to use of the pharmaceutical composition or pharmaceutical formulation in the manufacture of a medicament for treating a PDE3/4-mediated disease.

The pharmaceutical composition or the pharmaceutical formulation of the present invention has a dual bronchodilatory and anti-inflammatory effect, showing a high in vitro drug delivery efficiency with a fine particle fraction (FPF) of greater than 50%; good drug homogeneity with a relative standard deviation (RSD) of no greater than 5% for the mixing uniformity; and great formulation stability with no significant decrease in the fine particle fraction during storage and no significant increase in drug impurities.

The method for preparing the pharmaceutical composition or the pharmaceutical formulation of the present invention enables the obtainment of a pharmaceutical composition or pharmaceutical formulation with good stability, without the need for complex preparation processes.

The present invention provides a pharmaceutical composition, preferably a pharmaceutical composition for inhalation powder aerosol, comprising:
a) an active ingredient selected from the compound of Formula (I), or a stereoisomer, tautomer, pharmaceutically acceptable salt thereof;

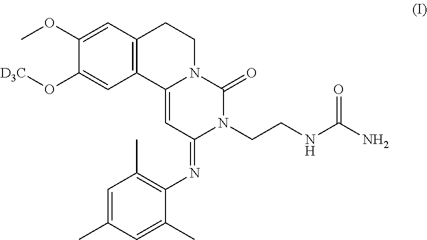 [SMILES: [2H]C([2H])([2H])Oc1cc2c(cc1OC)CCn1c-2c/c(=N\c2c(C)cc(C)cc2C)n(CCNC(N)=O)c1=O]

b) a pharmaceutical excipient;
wherein, the pharmaceutical excipient comprises one of a diluent and a lubricant or a combination of both; preferably, it simultaneously comprises a diluent and a lubricant.

The present invention provides a pharmaceutical composition, preferably a pharmaceutical composition for inhalation powder aerosol, comprising:
a) an active ingredient selected from the compound of Formula (I), or a stereoisomer, tautomer, or pharmaceutically acceptable salt thereof;
b) a pharmaceutical excipient;
wherein, the pharmaceutical excipient comprises one of a diluent and a lubricant or a combination of both. In some embodiments, the pharmaceutical composition for inhalation is a dry powder comprising a) the compound of Formula (I), or a stereoisomer, tautomer, or pharmaceutically acceptable salt thereof, and b) both a diluent and a lubricant.

In any of the embodiments of the present invention, as calculated on the basis of the free form of the active ingredient (i.e., the compound of Formula (I)), the amount of the active ingredient in the pharmaceutical composition is 0.001 g-30 g per 100 g of the pharmaceutical composition. In some embodiments, as calculated on the basis of the free form of the active ingredient (i.e., the compound of Formula (I)), the amount of the active ingredient in the pharmaceutical composition is 0.001 g-20 g per 100 g of the pharmaceutical composition. In some embodiments, as calculated on the basis of the free form of the active ingredient (i.e., the compound of Formula (I)), the amount of the active ingredient in the pharmaceutical composition is 0.1 g-15 g per 100 g of the pharmaceutical composition. In some embodiments, as calculated on the basis of the free form of the active ingredient (i.e., the compound of Formula (I)), the amount of the active ingredient in the pharmaceutical composition is 0.5 g-12 g per 100 g of the pharmaceutical composition.

In any of the embodiments of the present invention, the amount of the lubricant in the pharmaceutical composition is 0.001 g-5 g per 100 g of the pharmaceutical composition. In any of the embodiments of the present invention, the amount of the lubricant in the pharmaceutical composition is 0.01 g-2 g per 100 g of the pharmaceutical composition. In any of the embodiments of the present invention, the amount of the lubricant in the pharmaceutical composition is 0.02-2 g per 100 g of the pharmaceutical composition. In any of the embodiments of the present invention, the amount of the lubricant in the pharmaceutical composition is 0.1-2 g per 100 g of the pharmaceutical composition. In any of the embodiments of the present invention, the amount of the lubricant in the pharmaceutical composition is 0.12-2 g per 100 g of the pharmaceutical composition.

In any of the embodiments of the present invention, the lubricant in the pharmaceutical composition is selected from one or more of stearate, micronized silica gel, talcum powder, amino acid and a derivative thereof, polyethylene glycol, preferably stearate and micronized silica gel, preferably magnesium stearate and micronized silica gel, more preferably magnesium stearate. In any of the embodiments of the present invention, the lubricant in the pharmaceutical composition is selected from one or more of magnesium stearate, micronized silica gel, talcum powder, leucine, isoleucine, arginine, lysine, phenylalanine, tryptophan, methionine and polyethylene glycol. In any of the embodiments of the present invention, the lubricant in the pharmaceutical composition is magnesium stearate.

In any of the embodiments of the present invention, the diluent in the pharmaceutical composition is selected from one or more of lactose, mannitol, glycine, leucine, phospholipid, xylitol, and acacia gum. In any of the embodiments of the present invention, the diluent in the pharmaceutical composition is selected from one or more of lactose, mannitol and leucine. In any of the embodiments of the present invention, the diluent in the pharmaceutical composition is lactose. In some embodiments, the lactose is anhydrous lactose.

In any of the embodiments of the present invention, the amount of the diluent in the pharmaceutical composition is 70 g-99.99 g per 100 g of the pharmaceutical composition. In any of the embodiments of the present invention, the amount of the diluent in the pharmaceutical composition is 80 g-99.9 g per 100 g of the pharmaceutical composition. In any of the embodiments of the present invention, the amount of the diluent in the pharmaceutical composition is 85 g-99.5 g per 100 g of the pharmaceutical composition.

In some embodiments, the pharmaceutical composition of the present invention comprises an active ingredient that is the compound of Formula (I), or a stereoisomer, tautomer, pharmaceutically acceptable salt thereof, as well as a diluent and a lubricant;
the amount of the active ingredient is 0.001 g-30 g, preferably 0.001 g-20 g, preferably 0.1 g-15 g, more preferably 0.5 g-12 g per 100 g of the pharmaceutical composition;
the amount of the lubricant is 0.001 g-5 g, preferably 0.01 g-2 g, preferably 0.02-2 g, preferably 0.1-2 g, preferably 0.12-2 g per 100 g of the pharmaceutical composition; and
the amount of the diluent is 70 g-99.99 g, preferably 80 g-99.9 g, more preferably 85 g-99.5 g per 100 g of the pharmaceutical composition.

In some embodiments, the pharmaceutical composition of the present invention comprises an active ingredient that is the compound of Formula (I), or a stereoisomer, tautomer, pharmaceutically acceptable salt thereof, a diluent and a lubricant;
the amount of the active ingredient is present in an amount equivalent to about 0.1 g to about 5 g of the compound of Formula (I) per 100 g of the pharmaceutical composition;
the amount of the lubricant is about 0.02 g to about 2 g per 100 g of the pharmaceutical composition; and
the amount of the diluent is about 80 g to about 99.9 g per 100 g of the pharmaceutical composition.

In any of the embodiments of the present invention, the weight ratio of the active ingredient to the lubricant is 1:0.0001-1:10, preferably 1:0.001-1:5, more preferably 1:0.002-1:5, 1:0.002-1:3, 1:0.002-1:1, 1:0.002-1:0.5;
in any of the embodiments of the present invention, the weight ratio of the active ingredient to the diluent is 1:1-1:200, preferably 1:5-1:150, more preferably 1:7-1:100, 1:7-1:50.

In some embodiments, the pharmaceutical composition of the present invention comprises an the compound of Formula (I), lactose, and magnesium stearate, wherein the weight ratio of the compound of Formula (I) to the lactose is from about 1:10 to about 1:35, and the weight ratio of the compound of Formula (I) to the magnesium stearate is from about 1:0.005 to about 1:0.05.

In some embodiments, the pharmaceutical composition of the present invention comprises:
(i) an active ingredient that is the compound of Formula (I) in an amount of 0.5 g-12 g per 100 g of the pharmaceutical composition;
(ii) a lubricant that is selected from magnesium stearate in an amount of 0.01 g-2 g per 100 g of the pharmaceutical composition;
(iii) a diluent that is selected from lactose in an amount of 85 g-99.5 g per 100 g of the pharmaceutical composition.

In some embodiments, the pharmaceutical composition of the present invention comprises:
(i) the compound of Formula (I) in an amount of about 0.5 g to about 12 g per about 100 g of the pharmaceutical composition;
(ii) magnesium stearate in an amount of about 0.01 g to about 2 g per about 100 g of the pharmaceutical composition;
(iii) lactose in an amount of about 85 g to about 99.5 g per about 100 g of the pharmaceutical composition.

Particularly, in the embodiments of the present invention, each 100 g of the pharmaceutical composition comprises:

| | |
|---|---|
| micronized compound of Formula (I) | 6.82 g, |
| lactose | 93.06 g, |
| magnesium stearate | 0.12 g; |

or each 100 g of the pharmaceutical composition comprises:

| | |
|---|---|
| micronized compound of Formula (I) | 9.09 g, |
| lactose | 90.79 g, |
| magnesium stearate | 0.12 g; |

or each 100 g of the pharmaceutical composition comprises:

| | | |
|---|---|---|
| micronized compound of Formula (I) | 3.41 | g, |
| lactose | 96.47 | g, |
| magnesium stearate | 0.12 | g. |

In any of the embodiments of the present invention, the active ingredient is optionally subjected to micronization treatment; preferably, the method for the micronization treatment is selected from high-pressure homogenization, microfluidic homogenization, shear dispersion, ball milling, spray drying, freeze drying, freeze spray drying or jet milling, preferably spray drying, jet milling; more preferably jet milling.

In any of the embodiments of the present invention, the particle size distribution of the active ingredient has a D50 value of 0.5 μm-5 μm and a D90 value of no greater than 10 μm, preferably a D50 value of 1 μm-3 μm and a D90 value of no greater than 8 μm, preferably a D50 value of 1 μm-3 μm and a D90 value of no greater than 5 μm.

In some embodiments, the dry powder pharmaceutical composition comprises the compound of Formula (I), lactose, and magnesium stearate, wherein the particle size distribution of the compound of Formula (I) has a D50 value of about 0.5 μm to about 5 μm and a D90 value of no greater than about 10 μm. In some embodiments, the dry powder pharmaceutical composition comprises the compound of Formula (I), lactose, and magnesium stearate, wherein the particle size distribution of the compound of Formula (I) has a D50 value of about 1 μm to about 3 μm and a D90 value of no greater than about 8 μm. In some embodiments, the dry powder pharmaceutical composition comprises the compound of Formula (I), lactose, and magnesium stearate, wherein the particle size distribution of the compound of Formula (I) has a D50 value of about 1 μm to about 3 μm and a D90 value of no greater than about 5 μm.

In any of the embodiments of the present invention, the lactose is selected from one or both of coarse lactose and fine lactose having the following particle size ranges; preferably, the lactose is coarse lactose having the following particle size ranges; preferably, the lactose consists of both coarse lactose having the following particle size ranges and fine lactose having the following particle size ranges:
1) coarse lactose with a D90 of 60-240 μm, preferably 90-240 μm, preferably 80-220 μm, preferably 105-220 μm, preferably 120-200 μm, and more preferably 100-220 μm;
2) fine lactose with a D90 of less than 50 μm, preferably less than 40 μm, preferably less than 20 μm, preferably less than 15 μm, and more preferably less than 10 μm.

In any of the embodiments of the present invention, the dry powder formulations described herein comprise a coarse lactose and a fine lactose having the following particle size ranges:
1) coarse lactose with a D90 of about 60 μm to about 240 μm;
2) fine lactose with a D90 of about 50 μm or less than 50 μm.

In any of the embodiments of the present invention, the dry powder formulations described herein comprise a coarse lactose and a fine lactose having the following particle size ranges:
1) coarse lactose with a D90 of about 90 μm to about 240 μm;
2) fine lactose with a D90 of about 40 μm or less than 40 μm.

In any of the embodiments of the present invention, the dry powder formulations described herein comprise a coarse lactose and a fine lactose having the following particle size ranges:
1) coarse lactose with a D90 of about 80 μm to about 220 μm;
2) fine lactose with a D90 of about 20 μm or less than 20 μm.

In any of the embodiments of the present invention, the dry powder formulations described herein comprise a coarse lactose and a fine lactose having the following particle size ranges:
1) coarse lactose with a D90 of about 105 μm to about 220 μm;
2) fine lactose with a D90 of about 15 μm or less than 15 μm.

In any of the embodiments of the present invention, the dry powder formulations described herein comprise a coarse lactose and a fine lactose having the following particle size ranges:
1) coarse lactose with a D90 of about 120 μm to about 200 μm;
2) fine lactose with a D90 of about 10 μm or less than 10 μm.

In any of the embodiments of the present invention, the dry powder formulations described herein comprise a coarse lactose and a fine lactose having the following particle size ranges:
1) coarse lactose with a D90 of about 100 μm to about 220 μm;
2) fine lactose with a D90 of less than about 10 μm.

In any of the embodiments of the present invention, the dry powder formulations described herein comprise a coarse lactose and a fine lactose having the following particle size ranges:
1) coarse lactose with a D90 of about 100 to about 220 μm, a D50 of about 20 μm to about 90 μm, or a D90 of about 100 to about 220 μm and a D50 of about 20 μm to about 90 μm,
2) fine lactose with a D90 of about 10 μm or less than 10 μm, a D50 of about 5 μm or less than 5 μm, or both a D90 of about 10 μm or less than 10 μm and a D50 of about 5 μm or less than 5 μm,
In any of the embodiments of the present invention, the dry powder formulations described herein comprise a diluent and a lubricant, wherein the lubricant is magnesium stearate, and the diluent comprises both coarse lactose having the following particle size ranges and fine lactose having the following particle size ranges:

1) coarse lactose with a D90 of about 100 to about 220 μm, a D50 of about 20 μm to about 90 μm, or a D90 of about 100 to about 220 μm and a D50 of about 20 μm to about 90 μm,
2) fine lactose with a D90 of about 10 μm or less than 10 μm, a D50 of about 5 μm or less than 5 μm, or both a D90 of about 10 μm or less than 10 μm and a D50 of about 5 μm or less than 5 μm,

In any of the embodiments of the present invention, the weight percentage of the fine lactose relative to the total weight of the lactose is 1%-15%, for example 2%-15%, for example 2%-10%, for example 4%-8%, for example 4%, 5%, 6%, 7%, 8%.

In any of the embodiments of the present invention, the lactose is selected from one or more of Lactohale® 100, Lactohale® 200, Respitose® ML001, Lactohale® 206, InhaLac® 120, InhaLac® 140, InhaLac® 160, InhaLac® 400, Lactohale® 230, Lactohale® 300 and Respitose® SV003; preferably one or more of Lactohale® 200, Respitose® ML001, Lactohale® 206, InhaLac® 140, InhaLac® 400, Lactohale® 230, Lactohale® 300, and Respitose® SV003; and more preferably one or more of Lactohale® 200, Respitose® ML001, Lactohale® 230, Lactohale® 300, and Respitose® SV003.

In one aspect, the present invention provides a pharmaceutical formulation comprising the pharmaceutical composition described in any of the preceding embodiments;
in some embodiments, the amount of the active ingredient in a unit dose is 0.1 mg-10,000 mg, preferably 0.1 mg-100 mg, preferably 0.1 mg-10 mg, preferably 0.1 mg-5 mg, preferably 0.1 mg-3 mg, preferably 0.1 mg-2 mg, preferably 0.1 mg-1 mg, preferably 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 2 mg, 2.5 mg, 2.75 mg, 3 mg, 4 mg, 5 mg, 6 mg, 7 mg, 8 mg, 9 mg, 10 mg, 20 mg, 30 mg, 40 mg, 50 mg, 75 mg, 100 mg, 200 mg, 400 mg, 500 mg, 1000 mg.

In some embodiments, the pharmaceutical formulation is an inhalant, preferably an inhalation powder aerosol.

In some embodiments, the mass of the pharmaceutical composition per inhalation is 2 mg-100 mg, preferably 5 mg-50 mg.

In some embodiments, for the drug mixing uniformity of the pharmaceutical formulation of the present invention, the relative standard deviation (RSD) is not greater than 5%, preferably not greater than 3%.

In some embodiments, after the pharmaceutical formulation of the present invention is inhaled through a powder aerosol inhaler device, the fine particle fraction (FPF) is greater than 50%, for example greater than 55%, for example greater than 60%.

In some embodiments, the packaging form of the pharmaceutical formulation of the present invention is non-packaged, an aluminum-aluminum packaged capsule, an aluminum-plastic packaged capsule, preferably an aluminum-aluminum packaged capsule.

The present invention also provides a process for preparing the pharmaceutical formulation, comprising: filling the pharmaceutical composition, manually or using an automatic filling machine, into a capsule, an aluminum foil blister cavity, a powder aerosol inhaler device, preferably into a capsule, to obtain the pharmaceutical formulation.

The capsules used in the present invention are plant-based or animal-based capsules, preferably empty hydroxypropyl methylcellulose capsules, empty gelatin capsules.

The present invention provides use of the pharmaceutical composition of any of the preceding embodiments or the pharmaceutical formulation of any of the preceding embodiments in the manufacture of a medicament for treating a PDE3/4-mediated disease.

In some embodiments, the disease is selected from chronic obstructive pulmonary disease (COPD) and asthma.

The present invention also provides a method for treating a disease in a mammal comprising administering to a subject a therapeutically effective amount of the pharmaceutical composition described above or the pharmaceutical formulation described above, said disease being selected from COPD and asthma.

In one aspect, described herein is a method of treating an airway inflammatory disease in a human comprising administering to the human in need thereof one or two daily doses of the pharmaceutical composition described herein, wherein each dose of the pharmaceutical composition is administered by inhalation with a dry powder inhaler. In some embodiments, each dose of the pharmaceutical composition comprises the compound of Formula (I), or a pharmaceutically acceptable salt thereof, in an amount equivalent to about 0.1 mg to about 6 mg of compound of Formula (I).

In another aspect, described herein is a method of treating an airway inflammatory disease in a human comprising administering to the human in need thereof one or two daily doses of the pharmaceutical composition comprising micronized compound of Formula (I), or a pharmaceutically acceptable salt thereof, in an amount equivalent to about 0.1 mg to about 6 mg of compound of Formula (I) per dose, lactose, and magnesium stearate, wherein each dose of the pharmaceutical composition is administered by inhalation with a dry powder inhaler. In some embodiments, each dose of the pharmaceutical composition comprises the compound of Formula (I), or a pharmaceutically acceptable salt thereof, in an amount equivalent to about 0.1 mg, about 0.15 mg, about 0.20 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, or about 6 mg of the compound of Formula (I). In some embodiments, the weight of each dose is about 22 mg.

In some embodiments, two daily doses of the pharmaceutical composition are administered, and each dose of the pharmaceutical composition comprises the compound of Formula (I), or a pharmaceutically acceptable salt thereof, in an amount equivalent to about 0.1 mg, about 0.15 mg, about 0.20 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, or about 4 mg of the compound of Formula (I). In some embodiments, two daily doses of the pharmaceutical composition are administered, and each dose of the pharmaceutical composition comprises the compound of Formula (I), or a pharmaceutically acceptable salt thereof, in an amount equivalent to about 0.25 mg, about 0.5 mg, about 0.75 mg, about 1 mg, about 1.5 mg, about 2 mg, about 2.5 mg, about 3 mg, about 3.5 mg, or about 4 mg of the compound of Formula (I).

In some embodiments, one daily dose of the pharmaceutical composition is administered, and the daily dose of the pharmaceutical composition comprises the compound of Formula (I), or a pharmaceutically acceptable salt thereof, in an amount equivalent to about 0.1 mg, about 0.15 mg, about 0.20 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, or about 4 mg of the compound of Formula (I). In some embodiments, one daily dose of the pharmaceutical composition is administered, and the daily dose of the pharmaceutical composition comprises the compound of Formula (I), or a pharmaceutically acceptable salt thereof, in an amount equivalent to about 0.25 mg, about 0.5 mg, about 0.75 mg, about 1 mg, about 1.5 mg, about 2 mg, about 2.5 mg, about 3 mg, about 3.5 mg, or about 4 mg of the compound of Formula (I). In some embodiments, the weight of each dose is about 22 mg.

In one aspect, described herein is a method of treating an airway inflammatory disease in a human comprising administering to the human in need thereof one or two daily doses of the pharmaceutical composition described herein, wherein each dose of the pharmaceutical composition is administered by inhalation with a dry powder inhaler; wherein each dose comprises the compound of Formula (I), or a pharmaceutically acceptable salt thereof, in an amount equivalent to about 0.1 mg to about 3 mg of compound of Formula (I), a diluent and a lubricant, wherein the lubricant is magnesium stearate, and the diluent comprises both coarse lactose having the following particle size ranges and fine lactose having the following particle size ranges:
1) coarse lactose with a D90 of about 100 to about 220 μm, a D50 of about 20 μm to about 90 μm, or a D90 of about 100 to about 220 μm and a D50 of about 20 μm to about 90 μm,
2) fine lactose with a D90 of about 10 μm or less than 10 μm, a D50 of about 5 μm or less than 5 μm, or both a D90 of about 10 μm or less than 10 μm and a D50 of about 5 μm or less than 5 μm; and the particle size distribution of the compound of Formula (I), or a pharmaceutically acceptable salt thereof, has a D50 value of 0.5 μm-5 μm and a D90 value of no greater than 10 μm. In some embodiments, the particle size distribution of the compound of Formula (I), or a pharmaceutically acceptable salt thereof, has a D50 value of 1 μm-3 μm and a D90 value of no greater than 8 μm. In some embodiments, the particle size distribution of the compound of Formula (I), or a pharmaceutically acceptable salt thereof, has a D50 value of 1 μm-3 μm and a D90 value of no greater than 5 μm. In some embodiments, each dose comprises the compound of Formula (I). In some embodiments, each unit dose has a total weight of about 20 mg to about 30 mg. In some embodiments, each unit dose has a total weight of about 20 mg. In some embodiments, each unit dose has a total weight of about 21 mg. In some embodiments, each unit dose has a total weight of about 22 mg. In some embodiments, each unit dose has a total weight of about 23 mg. In some embodiments, each unit dose has a total weight of about 24 mg. In some embodiments, each unit dose has a total weight of about 25 mg. In some embodiments, each unit dose has a total weight of about 26 mg. In some embodiments, each unit dose has a total weight of about 27 mg. In some embodiments, each unit dose has a total weight of about 28 mg. In some embodiments, each unit dose has a total weight of about 29 mg. In some embodiments, each unit dose has a total weight of about 30 mg.

In some embodiments, the airway inflammatory disease is chronic obstructive pulmonary disease (COPD), bronchiectasis, asthma, acute respiratory distress syndrome, exercise-induced bronchoconstriction (EIB), chronic bronchitis, emphysema, alpha-1 antitrypsin deficiency, combined pulmonary fibrosis and emphysema (CPFE), or asthma-COPD overlap syndrome (ACOS). In some embodiments, the asthma is allergic asthma, aspirin-induced asthma, cough-variant asthma, exercise-induced asthma, nighttime asthma, steroid-resistant asthma, occupational asthma, eosinophilic asthma, neutrophilic asthma, or mixed eosinophilic and neutrophilic asthma.

In some embodiments, the airway inflammatory disease is chronic obstructive pulmonary disease (COPD). In some embodiments, treating COPD comprises lowering the annualized rate of pulmonary exacerbations, slowing the decline of lung function, prolonging the time to the first exacerbation, lowering the frequency of severe exacerbations, or combinations thereof, as compared to a human with COPD not being treated with the compound of formula (I). In some embodiments, the airway inflammatory disease is bronchiectasis.

In some embodiments, the DPI is breath actuated. In some embodiments, the DPI is a low-resistance breath-actuated DPI. In some embodiments, the DPI provides a pressure drop of up to 4 kPa at the peak inspiratory flow rate (PIF). In some embodiments, the DPI provides a pressure drop of up to 4 kPa at a peak inspiratory flow rate (PIF) of at least 50 L/min.

In some embodiments, the compound of Formula (I), or a stereoisomer, tautomer, or pharmaceutically acceptable salt thereof, in the pharmaceutical compositions, methods of treatment or use described herein is replaced with the compound of Formula (II), Formula (III), Formula (IV), Formula (V), or Formula (VI), or a stereoisomer, tautomer, or pharmaceutically acceptable salt thereof:

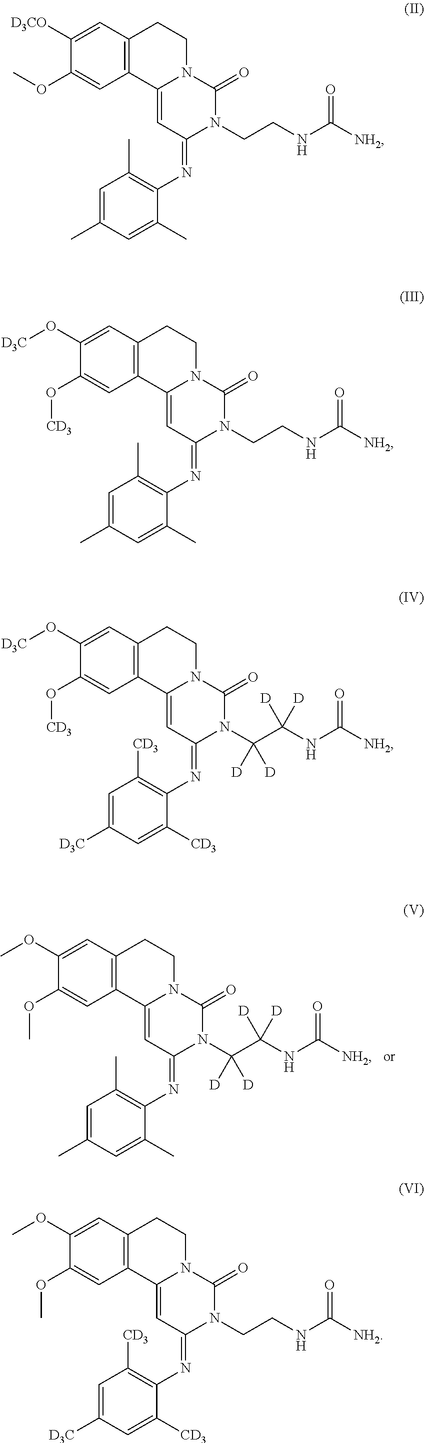

In some embodiments, the compound of Formula (I), or a pharmaceutically acceptable salt thereof, in the pharmaceutical compositions, methods of treatment or use described herein is replaced with the compound of Formula (II), or a pharmaceutically acceptable salt thereof. In some embodiments, the compound of Formula (I) in the pharmaceutical compositions, methods of treatment or use described herein is replaced with the compound of Formula (II).

In some embodiments, the compound of Formula (I), or a pharmaceutically acceptable salt thereof, in the pharmaceutical compositions, methods of treatment or use described herein is replaced with the compound of Formula (III), or a pharmaceutically acceptable salt thereof. In some embodiments, the compound of Formula (I) in the pharmaceutical compositions, methods of treatment or use described herein is replaced with the compound of Formula (III).

In some embodiments, the compound of Formula (I), or a pharmaceutically acceptable salt thereof, in the pharmaceutical compositions, methods of treatment or use described herein is replaced with the compound of Formula (IV), or a pharmaceutically acceptable salt thereof. In some embodiments, the compound of Formula (I) in the pharmaceutical compositions, methods of treatment or use described herein is replaced with the compound of Formula (IV).

In some embodiments, the compound of Formula (I), or a pharmaceutically acceptable salt thereof, in the pharmaceutical compositions, methods of treatment or use described herein is replaced with the compound of Formula (V), or a pharmaceutically acceptable salt thereof. In some embodiments, the compound of Formula (I) in the pharmaceutical compositions, methods of treatment or use described herein is replaced with the compound of Formula (V).

In some embodiments, the compound of Formula (I), or a pharmaceutically acceptable salt thereof, in the pharmaceutical compositions, methods of treatment or use described herein is replaced with the compound of Formula (VI), or a pharmaceutically acceptable salt thereof. In some embodiments, the compound of Formula (I) in the pharmaceutical compositions, methods of treatment or use described herein is replaced with the compound of Formula (VI).

In some embodiments, the compound of Formula (I), or a stereoisomer, tautomer, or pharmaceutically acceptable salt thereof, in the pharmaceutical compositions, methods of treatment or use described herein is replaced with the compound of Formula (VII), or a stereoisomer, tautomer, or pharmaceutically acceptable salt thereof:

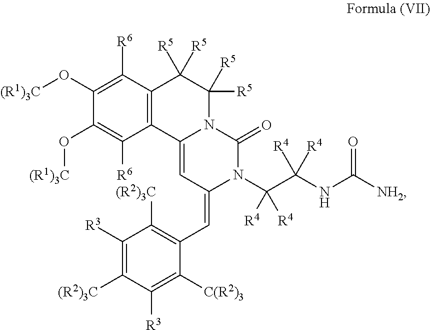

wherein each R¹, R², R³, R⁴, R⁵, and R⁶ is independently selected from hydrogen or deuterium, provided that at least 1 deuterium is present.

In some embodiments, each R¹, R², R³, R⁴, R⁵, and R⁶ is independently selected from hydrogen or deuterium, provided that at least 3 R¹ are deuterium.

In some embodiments, each R¹, R², R³, R⁴, R⁵, and R⁶ is independently selected from hydrogen or deuterium, provided that at least 3 R² are deuterium.

In some embodiments, each R¹, R², R³, R⁴, R⁵, and R⁶ is independently selected from hydrogen or deuterium, provided that at least 2 R⁴ are deuterium.

In some embodiments, each R¹, R², R³, R⁴, R⁵, and R⁶ is independently selected from hydrogen or deuterium, provided that at least 2 R⁵ are deuterium.

In some embodiments, each R¹, R², R³, R⁴, R⁵, and R⁶ is independently selected from hydrogen or deuterium, provided that at least 2 R⁶ are deuterium.

### CERTAIN TERMINOLOGY

The section headings used herein are for organizational purposes only and are not to be construed as limiting the subject matter described.

Unless otherwise stated, the terms used in the specification and claims have the following meanings:

As used herein, the terms “contain”, “contains”, “include”, “includes,” or “included” is not limiting.

The terms “treat”, “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, halting progression of the disease or condition, relieving a condition caused by the disease or condition, or stopping the symptoms of the disease or condition either prophylactically and/or therapeutically.

The term “acceptable” with respect to a formulation, composition or ingredient, as used herein, means having no persistent detrimental effect on the general health of the mammal being treated.

The terms “administer,” “administering”, “administration,” and the like, as used herein, refer to the methods that is used to enable delivery of compounds or compositions to the desired site of biological action. In some embodiments, the compounds and compositions described herein are administered by inhalation with a DPI inhaler.

The term “subject” or “patient” encompasses mammals. Examples of mammals include, but are not limited to, any member of the mammalian class: humans, non-human primates such as chimpanzees, and other apes and monkey species. In one aspect, the mammal is a human.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

In some embodiments, “therapeutically effective amount” refers to an amount of the compound of formula (I) that is administered to a human that is sufficient or effective to treat (e.g., halt, slow down the progression of, decrease the severity of) a respiratory disease associated with increased PDE3/4 activity, including alleviating symptoms of, or reducing the severity of symptoms of, such diseases. In some embodiments, “therapeutically effective amount” refers to an amount of the compound of formula (I) that is administered to a human with an inflammatory respiratory disease that is sufficient or effective to treat (e.g., halt, slow down the progression of, decrease the severity of) the inflammatory respiratory disease including alleviating symptoms of, or reducing the severity of symptoms of, the airway disease. In some embodiments, “therapeutically effective amount” refers to an amount of the compound of formula (I) that is administered to a human with COPD that is sufficient or effective to treat (e.g., halt, slow down the progression of, decrease the severity of) COPD in the human, including alleviating symptoms of, or reducing the severity of symptoms of, COPD.

The terms “about” or “approximately” mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value. In some embodiments, “about” means plus or minus 5% of the recited value, plus or minus 10% of the recited value, or plus or minus 15% of the recited value. In some embodiments, “about” means plus or minus 10% of the recited value.

The term “pharmaceutically acceptable” refers to compounds, materials, compositions, and/or dosage forms that, within sound medical judgment, are suitable for use in contact with human and animal tissues without excessive toxicity, irritation, allergic reactions, or other problems or complications, and are commensurate with a reasonable benefit/risk ratio.

“Pharmaceutically acceptable salt” refers to a salt that is safe, non-toxic, and neither biologically nor otherwise undesirable, and includes such salts that are pharmaceutically acceptable for veterinary use as well as human pharmaceutical use and possess the desired pharmacological activity.

“Stereoisomer” refers to isomers arising from different spatial arrangements of atoms in a molecule, including cis-trans isomers, enantiomers, and conformational isomers.

The term “pharmaceutical composition” refers to a mixture composed of one or more active ingredients of the present application, or a pharmaceutical combination thereof, together with pharmaceutically acceptable excipient(s). The purpose of a pharmaceutical composition is to facilitate the administration of a compound of the present application or a pharmaceutical combination thereof to a subject.

The term “pharmaceutically acceptable excipient” refers to those excipients that do not exert a significant irritant effect on an organism and do not impair the biological activity and properties of the active compound. Suitable excipients are well known to those skilled in the art, such as carbohydrates, waxes, water-soluble and/or water-swellable polymers, hydrophilic or hydrophobic materials, gelatin, oils, solvents, water, and the like.

The terms “unit dose”, “unit formulation” or “pharmaceutical composition per inhalation” bear their conventional meanings within the art, referring to the smallest administrable unit or smallest supplying unit or smallest packaging unit of a pharmaceutical product that is capable of being separated—for example, the unit dose of a tablet is one tablet, the unit dose of a capsule is one capsule, and the unit dose of an injection is one ampoule, and so forth.

Volume distribution refers to a statistical distribution weighted by the volume of an individual particle; it is typically used to describe the volumetric proportion occupied by particles within a sample.

Mixing uniformity (RSD): Mixing uniformity refers to the degree of homogeneity of a mixed material. Mixing uniformity RSD refers to the relative standard deviation of this uniformity. Relative standard deviation=[standard deviation (SD)/arithmetic mean of calculated result (X)]×100%.

The formulations described herein are suitable for inhalation. The inhalable formulations described herein include allow for rapid delivery of the compound of formula (I) into the circulatory system and/or target organ (e.g., the lungs) of a mammal in need thereof.

In certain embodiments, the inhalable formulation is in the form of a dry powder. In some instances, dry powders are one-phase solid-particle blends. In some instances, when actuated, dry powders are two-phase gas-solid systems wherein the dry powder is dispersed in air.

In some embodiments, dry powders contain at least one pharmaceutically acceptable excipient selected from pH-modifying agents, tonicity agents, propellants, preservatives, and surfactants.

In some embodiments, dry powders comprise micronized and/or nano-sized compound of formula (I) particles blended with larger carrier particles that prevent aggregation. The excipients and/or carriers in dry powders are endogenous to the lung and are easily metabolized or cleared.

To overcome the poor flow and high cohesion of fine drug particles, the drug particles are often blended with large carrier particles (e.g., lactose monohydrate). The drug particles adhere to the carrier during storage, which aids in handling. Upon inhalation, the drug must detach from the carrier. In some instances, dry powders contain the compound of formula (I) and lactose as a carrier. In some instances, dry powders comprise the compound of formula (I) and lactose monohydrate combined with magnesium stearate. Magnesium stearate acts as a “force control agent” to enhance aerosolization and improve stability by acting as a moisture barrier. In some instances, dry powders comprise the compound of formula (I) and starch, mannitol or glucose as a carrier. In some instances, dry powders are formulated as liposomes comprising phospholipids (e.g., phosphatidylcholine), cholesterol, or the like. A carrier particle having low hygroscopicity (e.g., lactose) prevents aggregation or caking due to absorption of moisture.

In some instances, a dry powder inhalable formulation described herein comprises nano-particles of the compound of formula (I). In some instances, dry powder inhalable formulations described herein comprise crystalline particles of the compound of formula (I). In some embodiments, dry powder inhalable formulations described herein comprise amorphous particles of the compound of formula (I).

In some embodiments, the inhalable formulation is administered with a dry powder inhaler (DPI), or a metered dose inhaler (MDI). In some embodiments, the inhalable formulation is administered with a dry powder inhaler (DPI).

In some instances, the dry powder inhalable formulations described herein are administered with a dry power inhaler (DPI). The medication is usually loaded as a capsule inside a chamber of the inhaler, and then delivered by a patient's inhalation, preferably a deep and forceful inhalation. Capsules (e.g., and cartridges of, such as, by way of example only, gelatin for use in an inhaler or insufflator may be formulated containing a dry powder formulation described herein. Medication in powder form, such as the dry powder formulations described herein, can be filled in either a capsule, blister, reservoir or cartridge, depending on the drug-device configuration. The device can be either an active or a passive device.

DPI devices include pre-metered (unit dose) DPI devices, device-metered (multi-dose) DPI devices, passive DPI devices, and active DPI devices. Pre-metered (unit dose) DPI devices include medication that is pre-filled into individual containers (e.g., capsules, blisters, or cartridges) and inserted into the device. A bulk reservoir of powder is housed within a device-metered (multi-dose) DPI, which meters out individual doses. Passive DPIs rely on the patient's inspiratory force to generate enough turbulent airflow to disperse the powder. Active DPIs use external energy (e.g., piezoelectric vibration) to aid in powder aerosolization.

In some embodiments, the dry powder inhalable formulations described herein are pre-filled into individual containers (e.g., capsules, blisters, or cartridges) that are inserted into the device. In some embodiments, the dry powder inhalable formulations described herein are pre-filled into individual capsules that are inserted into a passive DPI device.

The choice of container impacts powder stability and release, particularly for capsule-based devices (cDPIs). Gelatin capsules were traditionally used, but can become brittle in low-moisture environments (<30% RH). HPMC (hydroxypropyl methylcellulose) capsules are used more frequently due to their lower moisture content, superior piercing characteristics, and less sensitivity to moisture transfer from the formulation. Blister packs are used in multi-dose, pre-metered devices, offering high protection against moisture.

In some embodiments, the inhalable formulations described herein are administered with a metered dose inhaler (MDI). In some embodiments, the inhalable formulations described herein comprise a propellant and are pressure packaged for administration of the compound of formula (I) using pressurized aerosols. In some embodiments, the metered-dose inhaler comprises three major parts: a canister, a metering valve, and an actuator. In the case of a pressurized aerosol, the dosage unit may be determined by providing a valve to deliver a metered amount. In some instances, after pressing down the top of the canister, and actuating the MDI, a single metered dose containing the medication in the propellant is released, resulting an aerosol, which is then inhaled by the patient.

### Methods of Dosing and Treatment Regimens

In some embodiments, described herein is a method of treating a respiratory disease in a human comprising administering the compound of formula (I) to the human in need thereof. In some embodiments, described herein is a method of treating a respiratory disease in a human comprising administering a therapeutically amount of the compound of formula (I) to the human in need thereof. In some embodiments, the respiratory airway disease is chronic obstructive pulmonary disease (COPD), asthma, cystic fibrosis, non-cystic fibrosis bronchiectasis (NCFBE), adult respiratory distress syndrome, emphysema, chronic bronchiolitis, bronchitis, COVID-19, interstitial lung disease (ILD), lung cancer, pleurisy, pneumonia, pulmonary embolism, pulmonary fibrosis, pulmonary hypertension, or pulmonary sarcoidosis.

Chronic Obstructive Pulmonary Disease (COPD) mainly includes two types: emphysema and chronic bronchitis, which often coexist. These conditions cause long-term, progressive airflow obstruction usually caused by smoking or irritants. Emphysema damages air sacs, while chronic bronchitis causes airway inflammation and mucus. Emphysema damages the walls of the alveoli (air sacs) in the lungs, destroying the elasticity necessary for breathing and creating fewer, larger air spaces. Chronic bronchitis causes long-term inflammation of the bronchial tubes, leading to significant mucus production, a chronic productive cough, and frequent infections. Most people with COPD have a combination of both emphysema and chronic bronchitis.

Healthcare providers classify the severity of these types using the GOLD system: Grade 1 (Mild) COPD occurs when FEV1≥80% predicted; Grade 2 (Moderate) COPD occurs when 50%≤ FEV1<80% predicted; Grade 3 (Severe) COPD occurs when 30%≤FEV1<50% predicted; Grade 4 (Very Severe) COPD occurs when FEV1<30% predicted.

In some embodiments, the compound of formula (I) is used to treat an inflammatory respiratory disease in a human. In some embodiments, the compound of formula (I) is used to treat a neutrophil-driven respiratory disease in a human. In some embodiments, the compound of formula (I) is used to treat COPD in a human. In some embodiments, the compound of formula (I) is used to treat neutrophilic COPD in a human. In some embodiments, the compound of formula (I) is used to treat asthma in a human. In some embodiments, the compound of formula (I) is used to treat neutrophilic asthma in a human. In some embodiments, the compound of formula (I) is used to treat bronchiectasis in a human. In some embodiments, the compound of formula (I) is used to treat NCFBE in a human. In some embodiments, the compound of formula (I) is administered by inhalation. In some embodiments, the compound of formula (I) is administered by inhalation in the form of a dry powder with a dry powder inhaler. In some embodiments, a therapeutically effective amount of the compound of formula (I) is administered daily. In some embodiments, a therapeutically effective amount of the compound of formula (I) is administered once a day. In some embodiments, a therapeutically effective amount of the compound of formula (I) is administered twice a day. In some embodiments, a therapeutically effective amount of the compound of formula (I) is administered three times a day. In some embodiments, a therapeutically effective amount of the compound of formula (I) is administered once a day, twice a day, or three times a day.

In some embodiments, described herein is a method of treating COPD in a human comprising administering a pharmaceutical composition comprising the compound of formula (I) to the human in need thereof by inhalation with dry powder inhaler, wherein the pharmaceutical composition is a dry powder comprising the compound of formula (I) and one or more pharmaceutically acceptable excipients.

In some embodiments, treating COPD in a human with the compound of formula (I) comprises lowering the annualized rate of pulmonary exacerbations, slowing the decline of lung function, prolonging the time to the first exacerbation, lowering the frequency of severe exacerbations, or combinations thereof, as compared to a human with COPD not being treated with the compound of formula (I).

In some embodiments, treating NCFBE in a human with the compound of formula (I) comprises lowering the annualized rate of pulmonary exacerbations, slowing the decline of lung function, prolonging the time to the first exacerbation, lowering the frequency of severe exacerbations, or combinations thereof, compared to a human with NCFBE not being treated with the compound of formula (I).

In some embodiments, treating asthma in a human with the compound of formula (I) comprises lowering the annualized rate of pulmonary exacerbations, slowing the decline of lung function, prolonging the time to the first exacerbation, lowering the frequency of severe exacerbations, or combinations thereof, as compared to a human with asthma not being treated with the compound of formula (I).

As described herein, the compound of formula (I) is for use in a method of treating COPD. The compound may decrease the frequency and/or severity of COPD exacerbations in a patient suffering from COPD. The compound may decrease the frequency of COPD exacerbations. For instance, the patient may suffer two or fewer (for instance one or zero) COPD exacerbations per year while being treated with the compound, for instance as a maintenance therapy.

In some embodiments, the human with COPD experiences less than five COPD exacerbations per year while being treated with the compound of formula (I). In some embodiments, the human with COPD experiences less than three COPD exacerbations per year while being treated with the compound of formula (I). In some embodiments, the human with COPD experiences one or no COPD exacerbation per year while being treated with the compound of formula (I). In some embodiments, the human with COPD does not experience any COPD exacerbations per year while being treated with the compound of formula (I).

In some embodiments, COPD exacerbations are reduced by at least 20%, at least 25%, at least 30%, or at least 40%, while being treated with the compound of formula (I). In some embodiments, moderate COPD exacerbations are reduced by at least 20%, at least 25%, at least 30%, or at least 40%, while being treated with the compound of formula (I). In some embodiments, severe COPD exacerbations are reduced by at least 20%, at least 25%, at least 30%, or at least 40%, while being treated with the compound of formula (I).

In some embodiments, risks of COPD exacerbations are decreased by at least 20%, at least 25%, at least 30%, or at least 40%, vs. placebo after treatment with the compound of formula (I). In some instances, risk of COPD exacerbations is relative to placebo.

In some embodiments, the number of COPD exacerbations experienced by the human per year during treatment with the compound of formula (I) may be at least one, at least two, at least three, at least four, or at least times five fewer as compared to the number of COPD exacerbations experienced by a human prior to treatment with the compound (for instance per year in the one or two years prior to treatment with the compound of formula (I)).

In some embodiments, treatment with the compound of formula (I) increases the time to a first COPD exacerbation in the human. Accordingly, in some embodiments, the human may not have yet experienced a COPD exacerbation and the compound of formula (I) increases the time until the human experiences a first COPD exacerbation (i.e., the first COPD exacerbation is delayed). In some embodiments, the compound of formula (I) reduces the risk of COPD exacerbations in a COPD patient.

COPD exacerbations typically comprise one or more of the following symptoms: dyspnea (breathlessness), increased coughing, wheezing, sore throat, increased sputum volume, sputum purulence, a cold, and fever. Sputum purulence is a change in the color of spontaneously expectorated samples from uncolored to yellow green. In some embodiments, COPD exacerbation last for at least one day or at least two days. In some instances, the COPD exacerbation lasts for at least one day. In other instances, the COPD exacerbation lasts for at least two days.

In some embodiments, a COPD exacerbation comprises (i) worsening of one or more of the following symptoms for at least two consecutive days: dyspnea, sputum volume, and sputum purulence, and/or (ii) worsening of any one of the following symptoms for at least two consecutive days: sore throat, colds (nasal discharge and/or nasal congestion), fever (oral temperature >37.5° C) without other cause, and increased cough. In some embodiments, a COPD exacerbation comprises worsening of two or more of the major symptoms (dyspnea, sputum volume, and sputum purulence) for at least two consecutive days.

A COPD exacerbation may be a moderate COPD exacerbation or a severe COPD exacerbation. A moderate exacerbation is defined as worsening symptoms of COPD (as noted above) requiring a minimum of three days of treatment with oral/systemic corticosteroids and/or antibiotics. A severe exacerbation is defined as worsening symptoms of COPD (as defined above) requiring in-patient hospitalization. The compound of formula (I) may reduce the severity of COPD exacerbations in a COPD patient, and accordingly the compound of formula (I) may be for use in preventing severe COPD exacerbations in a COPD patient. For instance, the patient may experience no severe COPD exacerbations in the year following first administration of the compound of formula (I). The compound of formula (I) may be for preventing moderate COPD exacerbations in a COPD patient.

In some embodiments, treatment with the compound of formula (I) lengthens the time to a COPD exacerbation in the COPD patient. In some embodiments, treatment with the compound of formula (I) increases the time to an exacerbation by two or more months. In some embodiments, treatment with the compound of formula (I) lengthens the time to a COPD exacerbation by at least one month. In some embodiments, treatment with the compound of formula (I) lengthens the time to a COPD exacerbation by at least three, four, five, or six months. In some embodiments, the COPD patient is typically susceptible to COPD exacerbations. Typically, a patient susceptible to COPD exacerbations is a patient suffering from one or more comorbidities (other than COPD). Comorbidities of COPD include asthma, pulmonary hypertension, bronchiectasis, allergies, lung cancer, chest infection, cystic fibrosis, pulmonary fibrosis, pneumonia, hay fever, allergic rhinitis, bronchitis, emphysema, adult respiratory distress syndrome (ARDS), interstitial lung disease, and tuberculosis. In some embodiments, the asthma is allergic asthma, steroid resistant asthma, or severe asthma. In some embodiments, the COPD patient susceptible to COPD exacerbation has a chronic bronchitis etiology, an impaired lung function (such as 30 to 70% predicted Forced Expiratory Volume in the first second (FEV1), or has COPD symptoms despite use of long-acting muscarinic receptor antagonist (LAMA) or a long-acting beta-adrenergic receptor agonist (LABA) therapy.

In some embodiments, described herein is a method of treating COPD in a human diagnosed with both COPD and asthma comprising administering the compound of formula (I) the human in need thereof with a DPI inhaler. In some embodiments, described herein is a method of treating COPD in a human diagnosed with both COPD and bronchiectasis comprising administering the compound of formula (I) the human in need thereof with a DPI inhaler.

Other risk factors for COPD exacerbation include: high serum immunoglobulin (Ig), previous tuberculosis, severe airflow obstruction, chest infection, and one or more hospital admission for COPD exacerbations in the previous year.

In some cases, the human with COPD has suffered one or more COPD exacerbations in the year prior to initiating treatment with the compound of formula (I). For instance, the human may have suffered two or more COPD exacerbations in the year prior to initiating treatment with the compound of formula (I). The human may for instance have suffered from at least one severe COPD exacerbation (i.e. requiring hospital treatment) in the year prior to initiating treatment with the compound of formula (I). The human may have suffered one or more COPD exacerbations in the six months preceding first administration of the compound, or one or more COPD exacerbations during the month prior to initiating treatment with the compound of formula (I).

In some embodiments, described herein is a method of treating COPD in a human for whom inhaled corticosteroids are not a suitable treatment comprising administering the compound of formula (I) to the human in need thereof with a DPI inhaler. Inhaled corticosteroids include beclomethasone, budesonide, ciclesonide, flunisolide, fluticasone, mometasone, triamcinolone and salts or esters thereof.

In some embodiments, the COPD patient treated with the compound of formula (I) is male. In some embodiments, the COPD patient treated with the compound of formula (I) is female. In some embodiments, the COPD patient treated with the compound of formula (I) is 65 years old or older. In some embodiments, the COPD patient treated with the compound of formula (I) is 60 years old or older. In some embodiments, the COPD patient treated with the compound of formula (I) is 55 years old or older. In some embodiments, the COPD patient treated with the compound of formula (I) is 50 years old or older. In some embodiments, the COPD patient treated with the compound of formula (I) is 45 years old or older. In some embodiments, the COPD patient treated with the compound of formula (I) is 40 years old or older. In some embodiments, the COPD patient treated with the compound of formula (I) is already on therapy with a long-acting muscarinic antagonist (LAMA), a long-acting beta-agonist (LABA), or both.

Methods of treating any one of the respiratory diseases described herein in a human comprise administering the compound of formula (I) to the human in need thereof by inhalation. The compound of formula (I) is administered as a pharmaceutical composition with dry powder inhaler (DPI).

In some embodiments, described herein is a method of treating COPD in a human comprising administering a dry powder pharmaceutical composition comprising the compound of formula (I) to the human in need thereof by inhalation twice a day with dry powder inhaler. In some embodiments, a first dose of the compound of formula (I) is administered in the morning and a second dose of the compound of formula (I) is administered in the evening. In some embodiments, a first dose of the compound of formula (I) is administered in the morning within 3 hours following waking and a second dose of the compound of formula (I) is administered in the evening within 3 hours before bed. In some embodiments, the morning and evening doses are administered from about 10 to about 14 hours apart. In some embodiments, the morning and evening doses are administered about 12 hours apart.

Daily doses of the compound of formula (I) include about 0.1 mg to about 6 mg, about 0.25 mg, about 0.5 mg, about 0.75 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, or about 6.0 mg.

Typically, the compound of formula (I) is administered twice a day in two separate equal doses. For instance, the method comprises administering a first dose of the compound of formula (I) in an amount of about 0.25 mg, about 0.5 mg, about 0.75 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, or about 6.0 mg, and a second dose an amount equal to the first dose amount.

In some embodiments, about 0.25 mg, about 0.5 mg, about 0.75 mg, about 1.0 mg, about 1.5 mg, about 2.0 mg, about 2.5 mg, about 3.0 mg, about 3.5 mg, about 4.0 mg, about 4.5 mg, about 5.0 mg, about 5.5 mg, or about 6.0 mg of the compound of formula (I) is administered to the human twice a day.

In some embodiments, about 0.5 mg, about 0.75 mg, about 1.0 mg, about 1.5 mg, about 2.0 mg, about 2.5 mg, or about 3.0 mg of the compound of formula (I) is administered to the human twice a day.

In some embodiments, about 0.75 mg of the compound of formula (I) is administered to the human twice a day. In some embodiments, about 1.5 mg of the compound of formula (I) is administered to the human twice a day.

In some embodiments, about 0.25 mg of the compound of formula (I) is administered to the human twice a day. In some embodiments, about 3 mg of the compound of formula (I) is administered to the human twice a day.

In some embodiments, the compound of formula (I) is administered once or twice a day for at least 8 weeks. In some embodiments, the compound of formula (I) is administered once or twice a day for at least 16 weeks. In some embodiments, the compound of formula (I) is administered once or twice a day for at least 24 weeks. In some embodiments, the compound of formula (I) is administered once or twice a day for at least 1 year.

In any of the aforementioned embodiments are further embodiments comprising single administrations of the effective amount of the compound of formula (I), including further embodiments in which (i) the compound of formula (I) is administered once a day; or (ii) the compound of formula (I) is administered multiple times over the span of one day.

In any of the aforementioned embodiments are further embodiments comprising multiple administrations of the effective amount of the compound of formula (I), including further embodiments in which (i) the compound of formula (I) is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended, or the dose of the compound being administered is temporarily reduced; at the end of the drug holiday, dosing of the compound is resumed. In one embodiment, the length of the drug holiday varies from 2 days to 1 year.

In certain instances, it is appropriate to administer the compound of formula (I) in combination with one or more other therapeutic agents.

### DETAILED DESCRIPTION OF THE INVENTION

The following description illustrates with examples the technical problem to be solved, the technical solution, and the beneficial effect of the present invention. It should be understood that the specific examples described herein are for illustrating the present invention but are not intended to limit the present invention.

In the present invention, the active substance, i.e., the compound of Formula (I), is micronized, then mixed with a diluent and a lubricant, filled into a capsule, an aluminum foil blister cavity, or a powder aerosol inhaler device, and packaged to obtain the pharmaceutical composition.

Micronization is performed by high-pressure homogenization, microfluidic homogenization, shear dispersion, ball milling, or jet milling, preferably jet milling. Particle size distribution (PSD) is detected using a Sympatec HELOS laser particle sizer or a Mastersizer 3000 laser particle sizer and controlled for a D50 (the particle size corresponding to the 50th percentile of the cumulative particle size distribution) value of 0.5 μm to 5 μm and a D90 (the particle size corresponding to the 90th percentile of the cumulative particle size distribution) value of not greater than 10 μm, preferably a D50 value of 1 μm to 3 μm and a D90 value of not greater than 5 μm.

The mixing equipment is an agitating mixer, high-shear mixer, rotary mixer, or three-dimensional mixer, or other commonly used mixing equipment for solid formulations.

Capsules for filling with the pharmaceutical composition are plant-derived capsules (e.g., empty hydroxypropyl methylcellulose capsules) or animal-derived capsules (e.g., empty gelatin capsules) of Size 3 or other suitable sizes.

Lactose may be selected from one or more of conventional lactoses, such as Lactohale® 100, Lactohale® 200, Respitose® ML001, Lactohale® 206, InhaLac® 120, InhaLac® 140, InhaLac® 160, InhaLac® 400, Lactohale® 230, Lactohale® 300, Respitose® SV003, etc. Other excipients are selected from pharmaceutical excipients commonly used in solid formulations.

Packaging materials are selected from cold stamping formed polyamide/aluminum/polyvinyl chloride medicinal composite hard sheets and polyester/aluminum medicinal composite films, and other packaging materials commonly used in solid formulations.

Drug delivery devices may be selected from the powder aerosol cartridge (a pharmaceutical powder inhaler) described in Patent CN104644435B, as well as from the “HandiHaler®” capsule-type pharmaceutical powder inhaler, “Breezhaler®” capsule-type pharmaceutical powder inhaler, “RS01” capsule-type pharmaceutical powder inhaler, “Turbuhaler®” reservoir-type pharmaceutical powder inhaler, “Accuhaler®” multi-dose aluminum foil blister cavity-type pharmaceutical powder inhaler, and various inhaler devices utilizing similar principles.

The clinical effectiveness of inhaled therapy relies on its ability to deliver drug directly to the intended site and avoiding systemic side effects. Patients' inhalation flow pattern can significantly influence the performance of an inhaler, thus impact effectiveness of the inhalation therapy.

The inspiratory airflow generated by the patient represents the only active force able to produce the micro-dispersion of the powder formulation for inhalation. One of the most important characteristics of micro-dispersed particles generated after inspiration is their particle size. Inhaled drug particles will deposit in different regions of the respiratory tract according to their particle size: particles of 1-5 μm will deposit in the alveolar region—the target area of therapeutic application—while particles >5 μm will predominately deposit in the oropharynx and upper airways. Particles of 8-10 μm will predominately deposit in the throat.

In order to de-agglomerate powder particles from a bond on larger carrier molecules (such as lactose) into a respirable dose, a sufficient flow rate must be achieved in the DPI device. On the other hand, stronger air flows cause a higher grade of impaction, resulting in higher rates of oropharyngeal deposition. Therefore, lung deposition in most DPIs depends considerably on the patients' inspiratory flow rate and the particular device's intrinsic resistance. The intrinsic resistance to airflow through the device is an important determinant of the final flow rate resulting in the inhaler.

The efficacy of DPIs depends on the strength and duration of a single inhalation by the user. The duration of the test is set on the basis of the total air volume typically inhaled in one adult breath, adjusted to be four litres in the case of the EurPh and two litres in the case of the USP. DPI devices have different intrinsic degrees of resistance to flow, i.e. some require more effort to inhale than others. A low-resistance device presents less resistance to airflow, meaning that it may be easier to use and therefore more effective for patients. Conversely, in high-resistance devices, patients need to apply greater effort to generate the necessary inspiratory flow to allow for an optimum drug delivery.

Patient characteristics such as age, gender and disease severity can affect the performance of the inhalers; studies have reported that increasing age and COPD severity, may reduce patients' inhalation capability. It is generally accepted that for a set inspiratory effort, the inhalation flows when using a low resistance DPI are expected be greater than when using a DPI with a higher resistance.

The mechanism of breath actuation in DPIs requires patients to generate airflow against the intrinsic airflow resistance of the inhaler. The reliance on patient-generated flow rate for effective inhalation can be minimized when using an inhaler with low intrinsic airflow resistance, so that patients need to exert lower inspiratory effort to generate sufficient and sustained airflow. Patients with respiratory disease (e.g., COPD or asthma) can inhale more comfortably though low resistance inhaler.

At pressure drop of up to 4 kPa (equivalent to 41 cmH₂O), patients are able to inhale comfortably through a DPI. The inspiratory efforts required by patients' becomes an important consideration in treatment decisions, when a minimum inhalation flow through a specific DPI is critical for efficient de-aggregation of drug particles, which is not the case with low resistance DPIs. Most low-resistance DPIs are capable of providing consistent dose delivery performance using airflow rates between about 50 and about 100 L/min.

Some COPD patients may have difficulty with the effort required to generate sufficient inspiratory flow through a high-resistance inhaler because of loss of muscle mass and/or COPD severity. In some embodiments, low-resistance DPIs and/or medium-resistance DPIs are preferred among these patients. In some embodiments, low-resistance DPIs are preferred among COPD patients. In some embodiments, medium resistance DPIs allow for the generation of necessary turbulence for drug delivery even with reduced patient effort.

The Aerodynamic Particle Size Distribution (APSD) of the pharmaceutical composition is tested using the next generation impactor (NGI).

### EXAMPLES

### Examples 1-5: Pharmaceutical Compositions Prepared with Different Diluents and Lubricants

### The Preparation Method for the Pharmaceutical Composition Comprises:

a. weighing out the compound of Formula (I) exceeding the prescribed amount and pulverizing it with a jet mill to obtain the micronized compound of Formula (I);
b. first mixing step: weighing out the prescribed amount of diluent and the prescribed amount of lubricant and mixing them with a three-dimensional mixer to obtain Mixture \textcircled{1};
c. second mixing step: adding Mixture \textcircled{1} and the micronized compound of Formula (I) to a mixing vessel, mixing them with a three-dimensional mixer, and passing the mixture through a 60-mesh pharmacopoeia sieve after mixing to obtain a total mixed pharmaceutical powder;
d. filling 23 mg of the total mixed pharmaceutical powder into an empty gelatin capsule;
e. packaging the capsule with aluminum-aluminum packaging to obtain the formulations of Examples 1-5.

Specifically, Example 1 was obtained by mixing lactose, magnesium stearate and the micronized compound of Formula (I),

Example 2 was obtained by mixing lactose, micronized silica gel and the micronized compound of Formula (I),

Example 3 was obtained by mixing anhydrous lactose, magnesium stearate and the micronized compound of Formula (I),

Example 4 was obtained by mixing mannitol, magnesium stearate and the micronized compound of Formula (I),

Example 5 was obtained by mixing leucine, magnesium stearate and the micronized compound of Formula (I).

The following table shows the mass of each component per 100 g of the composition.

Specifically, the lactose is a composition of Lactose 1 and Lactose 2. The particle size of Lactose 1 in the formulations is such that its D90 falls within the range of 70 to 120 μm. The particle size of Lactose 2 in the formulations is such that its D90 falls within the range of 6 to 35 μm. Lactose 2 accounts for 8% of the total weight of lactose by weight percentage.

| Item | | Example 1 | Example 2 | Example 3 | Example 4 | Example 5 |
|---|---|---|---|---|---|---|
| Active | Micronized compound of | 6.82 | 6.82 | 6.82 | 6.82 | 6.82 |
| ingredient | Formula (I) (g) | | | | | |
| Diluent | Lactose (g) | 93.08 | 93.08 | / | / | / |
| | Anhydrous lactose (g) | / | / | 93.08 | / | / |
| | Mannitol (g) | / | / | / | 93.08 | / |
| | Leucine (g) | / | / | / | / | 93.08 |
| Lubricant | Magnesium stearate (g) | 0.1 | / | 0.1 | 0.1 | 0.1 |
| | Micronized silica gel (g) | / | 0.1 | / | / | / |
| Measurement | Amount delivered out of | 1187 | 1167 | 1105 | 1126 | 1208 |
| | the inhaler device (μg)¹ | | | | | |
| | Fine particle dose FPD (μg)² | 760 | 604 | 619 | 619 | 616 |
| | Fine particle fraction FPF | 64 | 52 | 56 | 55 | 51 |
| | (%)³ | | | | | |

¹Amount delivered out of the inhaler device: the amount of drug released from the inhaler device, usually the cumulative mass of the drug collected from all parts of the next generation impactor (NGI);
²Fine particle dose (FPD): the cumulative mass of the fraction smaller than 5 μm in the distribution as collected from the next generation impactor (NGI); and
³Fine particle fraction (FPF): the proportion of the fine particle dose to the amount of drug delivered out of the inhaler device.

Due to small particle size, high adhesion, and poor flowability, it is difficult for the micronized compound of Formula (I) to be prepared into a uniform drug-excipient composition, which affects the single-dose accuracy. Examples 1-5 all obtained uniform pharmaceutical compositions with the addition of a diluent and a lubricant.

### Examples 6-11: Different Formulations with Lactose as the Diluent and Magnesium Stearate as the Lubricant

The preparation method for the composition comprises:
a. first mixing step: weighing out the prescribed amount of lactose and the prescribed amount of magnesium stearate, and mixing them with a three-dimensional mixer to obtain Mixture \textcircled{1};
b. second mixing step: dividing the amount of Mixture \textcircled{1} into two portions, adding one portion of Mixture \textcircled{1}, the micronized compound of Formula (I), and the other portion of Mixture \textcircled{1} sequentially to a mixing vessel, mixing them with a three-dimensional mixer, and passing the mixture through a 60-mesh pharmacopoeia sieve after mixing to obtain a total mixed pharmaceutical powder;
testing bulk density, tapped density, compressibility index, and mixing uniformity of the powder.

The mass of each component per 100 g of the composition is shown for the following Examples.

Example 6 was obtained by mixing lactose and an amount of the micronized compound of Formula (I); and a portion of the pharmaceutical powder was filled into empty gelatin capsules to achieve a weight of 21 mg of total mixed pharmaceutical powder per capsule, for which aluminum-aluminum packaging was used.

Examples 7-11 were obtained by mixing lactose, magnesium stearate and the micronized compound of Formula (I).

The following table shows the mass of each component per 100 g of the composition.

Specifically, the lactose is a composition of Lactose 1 and Lactose 2. The particle size of Lactose 1 in the formulations is such that its D90 falls within the range of 70 to 120 μm. The particle size of Lactose 2 in the formulations is such that its D90 falls within the range of 6 to 35 μm. Lactose 2 accounts for 8% of the total weight of lactose by weight percentage.

| | | Example | Example | Example | Example | Example | Example |
|---|---|---|---|---|---|---|---|
| Item | | 6 | 7 | 8 | 9 | 10 | 11 |
| Ingredient | Micronized compound of | 12 | 12 | 12 | 12 | 12 | 12 |
| | Formula (I) (g) | | | | | | |
| | Lactose (g) | 88 | 87.995 | 87.96 | 87.85 | 87 | 86 |
| | Magnesium stearate (g) | 0 | 0.005 | 0.04 | 0.15 | 1 | 2 |
| Measurement | Bulk density (g/ml) | 0.589 | 0.586 | 0.629 | 0.652 | 0.664 | 0.675 |
| | Tapped density (g/ml) | 0.998 | 0.977 | 1.015 | 1.035 | 1.038 | 1.055 |
| | Compressibility index (%) | 41 | 40 | 38 | 37 | 36 | 36 |
| | Mixing uniformity RSD (%) | 9 | 6 | 4 | 2 | 2 | 2 |

Bulk density: The mass of powder per unit volume when the powder sample naturally fills a specified container.
Tapped density: The filling density of powder in a tapped state. The tapped state is the state of the powder column after tapping down the powder sample in the container at a specific frequency until the volume no longer changes.
Compressibility index (%) is calculated by the formula: 100(V₀ − V_{F})/V₀, wherein: V₀ is the apparent volume in the loose state; and V_{F} is the tapped volume.

Example 6 contains the micronized compound of Formula (I) but not magnesium stearate, showing a compressibility index of 41% and displaying poor flowability and mixing uniformity. Examples 7-11 contain the micronized compound of Formula (I) and magnesium stearate, showing a compressibility index of 36% to 40%. The addition of an amount of magnesium stearate to the composition could significantly improve the blend uniformity of the powder blend containing the micronized compound of Formula (I), with an RSD of less than 5%.

### Examples 12-20: Formulations Containing 0 g, 0.02 g, 0.12 g, 1 g, and 2 g of Magnesium Stearate Per 100 g of the Composition

The preparation method for the composition comprises:
a. adding the prescribed amount of lactose, magnesium stearate, and micronized compound of Formula (I) sequentially to an agitating mixer, and passing them through a 60-mesh pharmacopoeia sieve after mixing to obtain a total mixed pharmaceutical powder;
b. filling 21 mg of the total mixed pharmaceutical powder into an empty hydroxypropyl methylcellulose capsule;
c. packaging the capsule with aluminum-aluminum packaging to obtain the formulations of Examples 12-20.

Specifically, Example 12 was obtained by mixing a low content of the micronized compound of Formula (I) and the prescribed amount of lactose; Examples 13-16 were obtained by mixing a low content of the micronized compound of Formula (I) with a different prescribed amount of magnesium stearate and corresponding prescribed amount of lactose, respectively; Examples 17-20 were obtained by mixing a high content of the micronized compound of Formula (I) with a different prescribed amount of magnesium stearate and corresponding prescribed amount of lactose, respectively.

The following table shows the mass of each component per 100 g of the composition.

Specifically, the lactose is a composition of Lactose 1 and Lactose 2. The particle size of Lactose 1 in the formulations is such that its D90 falls within the range of 70 to 120 μm. The particle size of Lactose 2 in the formulations is such that its D90 falls within the range of 6 to 35 μm. Lactose 2 accounts for 8% of the total weight of lactose by weight percentage.

| | | Ex. | Ex. | Ex. | Ex. | Ex. | Ex. | Ex. | Ex. | Ex. | Ex. |
|---|---|---|---|---|---|---|---|---|---|---|---|
| Item | | 12 | 13 | 14 | 15 | 16 | 6 | 17 | 18 | 19 | 20 |
| Ingredient | Micronized compound of | 0.5 | 0.5 | 0.5 | 0.5 | 0.5 | 12 | 12 | 12 | 12 | 12 |
| | Formula (I) (g) | | | | | | | | | | |
| | Lactose (g) | 99.5 | 99.48 | 99.38 | 98.5 | 97.5 | 88 | 87.98 | 87.88 | 87 | 86 |
| | Magnesium stearate (g) | 0 | 0.02 | 0.12 | 1 | 2 | 0 | 0.02 | 0.12 | 1 | 2 |
| Measurement | Mixing uniformity RSD (%) | 8 | 4 | 2 | 2 | 2 | 9 | 4 | 3 | 3 | 2 |
| | Amount delivered out of | 89 | 88 | 88 | 89 | 89 | 1934 | 2065 | 2018 | 2014 | 2031 |
| | the inhaler device (μg) | | | | | | | | | | |
| | Fine particle dose FPD (μg) | 36 | 45 | 51 | 52 | 52 | 832 | 1053 | 1271 | 1269 | 1280 |
| | Fine particle fraction FPF | 40 | 51 | 58 | 58 | 58 | 43 | 51 | 63 | 63 | 63 |
| | (%) | | | | | | | | | | |

In comparison, Example 12 and Example 6, which are pharmaceutical compositions containing no magnesium stearate, showed poorer mixing uniformity and lower fine particle fraction. For compositions containing a low or high content of the micronized compound of Formula (I), those comprising the micronized compound of Formula (I), lactose, and magnesium stearate had significantly increased FPF compared to those comprising the micronized compound of Formula (I) and lactose. Therefore, magnesium stearate improved the aerodynamic distribution and increased the effective pulmonary deposition dose of the compound of Formula (I).

### Examples 21-25: Formulations Containing 0.12 g of Magnesium Stearate and 1.71 g, 3.41 g, 5.11 g, 6.82 g, and 9.09 g of the Micronized Compound of Formula (I) Per 100 g of the Composition

The preparation method for the composition comprises:
a. adding the prescribed amount of lactose, magnesium stearate, and micronized compound of Formula (I) sequentially to an agitating mixer, and passing them through a 60-mesh pharmacopoeia sieve after mixing to obtain a total mixed pharmaceutical powder;
b. filling 21 mg of the total mixed pharmaceutical powder into an empty hydroxypropyl methylcellulose capsule;
c. packaging the capsule with aluminum-aluminum packaging to obtain the formulations of Examples 21-25.

The following table shows the mass of each component per 100 g of the composition.

Specifically, the lactose is a composition of Lactose 1 and Lactose 2. The particle size of Lactose 1 in the formulations is such that its D90 falls within the range of 100 to 220 μm. The particle size of Lactose 2 in the formulations is such that its D90 falls within the range of 6 to 10 μm. Lactose 2 accounts for 4% of the total weight of lactose by weight percentage.

| | | Example | Example | Example | Example | Example | Example |
|---|---|---|---|---|---|---|---|
| Item | | 21 | 22 | 23 | 24 | 25 | 18 |
| Ingredient | Micronized compound of | 1.71 | 3.41 | 5.11 | 6.82 | 9.09 | 12 |
| | Formula (I) (g) | | | | | | |
| | Lactose (g) | 98.17 | 96.47 | 94.77 | 93.06 | 90.79 | 87.88 |
| | Magnesium stearate (g) | 0.12 | 0.12 | 0.12 | 0.12 | 0.12 | 0.12 |
| Measurement | Mixing uniformity RSD (%) | 2 | 2 | 2 | 2 | 3 | 3 |
| | Amount delivered out of | 312 | 582 | 858 | 1106 | 1525 | 2018 |
| | the inhaler device (μg) | | | | | | |
| | Fine particle dose FPD (μg) | 184 | 361 | 515 | 730 | 976 | 1271 |
| | Fine particle fraction FPF | 59 | 62 | 60 | 66 | 64 | 63 |
| | (%) | | | | | | |

The compositions comprising the micronized compound of Formula (I), lactose, and magnesium stearate had good mixing uniformity, and compositions with different percentages of the micronized compound of Formula (I) all had high FPF (greater than 50%).

The pharmaceutical composition of Example 24 was filled into hydroxypropyl methylcellulose capsules with filling weights of 2 mg, 5 mg, 10 mg, 20 mg, 35 mg, 50 mg, and 100 mg, respectively. It was found that capsules containing larger amounts of the composition had higher FPF.

| Example 24 | 2 mg | 5 mg | 10 mg | 20 mg | 35 mg | 50 mg | 100 mg |
|---|---|---|---|---|---|---|---|
| Fine particle fraction FPF (%) | 52 | 54 | 56 | 65 | 63 | 65 | 65 |

The pharmaceutical composition of Example 24 was filled into empty hydroxypropyl methylcellulose capsules and tested using the powder aerosol cartridge (a pharmaceutical powder inhaler) described in Patent CN104644435B, “HandiHaler®” capsule-type pharmaceutical powder inhaler, “Breezhaler®” capsule-type pharmaceutical powder inhaler, or the pharmaceutical powder was tested using the “Turbuhaler®” reservoir-type pharmaceutical powder inhaler, “Accuhaler®” multi-dose aluminum foil blister cavity-type pharmaceutical powder inhaler and RS01 hand held capsule-based dry powder inhaler. FPFs were all above 50% as well.

| | Powder aerosol | | | | | |
|---|---|---|---|---|---|---|
| | cartridge (See | | | | | |
| Example 24 | CN104644435B) | HandiHaler ® | Breezhaler ® | Turbuhaler ® | Accuhaler ® | RS01 |
| Fine particle | 64 | 58 | 63 | 60 | 60 | 58.5 |
| fraction FPF (%) | | | | | | |

The pharmaceutical composition of Example 22 was filled into empty hydroxypropyl methylcellulose capsules at filling weights of 15 mg, 25 mg, and 35 mg, respectively; it was found that, at these filling weights, the FPF of the compositions exhibited good stability.

| | Filling | Filling | Filling |
|---|---|---|---|
| Example 22 | weight 15 mg | weight 25 mg | weight 35 mg |
| Fine particle | 58 | 60 | 61 |
| fraction FPF (%) | | | |

The pharmaceutical composition of Example 22 was filled into empty hydroxypropyl methylcellulose capsules and empty gelatin capsules of different brands at a filling weight of 21 mg, respectively. It was found that the different capsules had a minor impact on the fine particle fraction of the composition, and the measured FPF stability was found to be robust.

| | Hydroxypropyl | Hydroxypropyl | Hydroxypropyl | |
|---|---|---|---|---|
| | methylcellulose | methylcellulose | methylcellulose | Gelatin |
| Example 22 | capsules A | capsules B | capsules C | capsules A |
| Fine particle fraction FPF (%) | 62 | 58 | 60 | 58 |

When Example 22 was used in conjunction with pharmaceutical powder inhalers from different batches, it was found that the different batches of pharmaceutical powder inhalers had a minor impact on the fine particle fraction of the composition, and the measured FPF stability was found to be robust.

| | Pharmaceutical | Pharmaceutical | Pharmaceutical |
|---|---|---|---|
| | powder inhaler | powder inhaler | powder inhaler |
| Example 22 | batch 1 | batch 2 | batch 3 |
| Fine particle | 58 | 62 | 59 |
| fraction FPF (%) | | | |

### Examples 26-30

Examples 26-30 were obtained by using different pulverization pressures in jet milling to treat the compound of Formula (I) to give the micronized compound of Formula (I) with different particle size distributions, weighing out the prescribed amount of the micronized compound of Formula (I), and mixing it with the prescribed amount of magnesium stearate and the prescribed amount of lactose, respectively.

Specifically, the lactose is a composition of Lactose 1 and Lactose 2. The particle size of Lactose 1 in the formulations is such that its D90 falls within the range of 100 to 220 μm. The particle size of Lactose 2 in the formulations is such that its D90 falls within the range of 6 to 10 μm. Lactose 2 accounts for 4% of the total weight of lactose by weight percentage.

| Ingredient | Example 26 | Example 27 | Example 28 | Example 29 | Example 30 |
|---|---|---|---|---|---|
| Micronized compound | 6.82 | | | | |
| of Formula (I) (g) | | | | | |
| Lactose (g) | 93.06 | | | | |
| Magnesium stearate | 0.12 | | | | |
| (g) | | | | | |

The composition was prepared by the following process:
a. first mixing step: weighing out the prescribed amount of lactose and the prescribed amount of magnesium stearate, and mixing them with a three-dimensional mixer to obtain Mixture 1;
b. second mixing step: adding Mixture \textcircled{1} and the micronized compound of Formula (I) to a high-shear mixer for mixing to obtain a total mixed pharmaceutical powder;
c. filling 21 mg of the total mixed pharmaceutical powder into an empty gelatin capsule;
d. packaging the capsule with aluminum-aluminum packaging to obtain the formulations of Examples 26-30.

The above example formulations were tested in vitro for the delivery efficiency of inhalation, with results shown as follows.

| | Particle size | Particle size | Amount | | |
|---|---|---|---|---|---|
| | D50 (μm) of the | D90 (μm) of the | delivered out | | Fine particle |
| | compound of | compound of | of the inhaler | Fine particle | fraction FPF |
| Example | Formula (I) | Formula (I) | device (μg) | dose FPD (μg) | (%) |
| Example 26 | 1.3 | 2.7 | 1127 | 733 | 65 |
| Example 27 | 1.5 | 3.5 | 1160 | 708 | 61 |
| Example 28 | 2.2 | 4.2 | 1075 | 613 | 57 |
| Example 29 | 2.5 | 4.9 | 1100 | 572 | 52 |
| Example 30 | 3.6 | 8.5 | 1098 | 494 | 45 |

As demonstrated by Examples 26-30, the particle size with a D50 value of 1 μm-3 μm and a D90 value of no greater than 5 μm enabled a fine particle fraction of greater than 50%.

### Examples 31-33

They were obtained by mixing the same prescribed amount of magnesium stearate with a different prescribed amount of the micronized compound of Formula (I) and corresponding prescribed amount of lactose, respectively.

Specifically, the lactose is a mixture of Respitose® ML001 and Lactohale® 300, wherein Lactohale® 30 accounts for 4% of the total weight of lactose by weight percentage.

| Ingredient | Example 31 | Example 32 | Example 33 |
|---|---|---|---|
| Micronized compound of | 6.82 | 9.09 | 3.41 |
| Formula (I) (g) | | | |
| Lactose (g) | 93.06 | 90.79 | 96.47 |
| Magnesium stearate (g) | 0.12 | 0.12 | 0.12 |

The following preparation process was used:
a. first mixing step: weighing out one-twentieth of the prescribed amount of lactose, magnesium stearate, and the micronized compound of Formula (I), and mixing them with a three-dimensional mixer to obtain Mixture \textcircled{1};
b. second mixing step: adding the amount of Mixture \textcircled{1} and the remaining prescribed amount of lactose to a high-shear mixer for mixing to obtain a total mixed pharmaceutical powder;
c. filling 23 mg of the pharmaceutical composition powder into an empty gelatin capsule;
d. packaging the capsule with aluminum-aluminum packaging to obtain the formulations of Examples 31-33.

After aluminum-aluminum packaging, Examples 31-33 showed no significant increase in drug impurities and no significant decrease in fine particle fraction under a long-term condition of 30° C/65% RH and an accelerated condition of 40° C/75% RH. Therefore, the compositions of the present invention have excellent formulation stability. As a comparison, the composition containing no magnesium stearate in Example 6 showed a significant decrease in fine particle dose and fraction under the accelerated condition of 40° C/75% RH and exhibited instability.

| | | Related substances (%) | | | | |
|---|---|---|---|---|---|---|
| | | RRT | | Amount | Fine | Fine |
| | | approx. | | delivered out | particle | particle |
| | | 1.20 ≤ | Total | of the inhaler | dose FPD | fraction |
| Item | | 1.0% | impurities ≤3.0% | device (μg) | (μg) | FPF (%) |
| Example 6 (with | 0 month | 0.4 | 0.53 | 1934 | 832 | 43 |
| aluminum- | Long-term 3 months | 0.41 | 0.55 | 1987 | 755 | 38 |
| aluminum | Accelerated 1 month | 0.4 | 0.61 | 1922 | 692 | 36 |
| packaging) | | | | | | |
| Example 31 | 0 month | 0.41 | 0.54 | 1110 | 710 | 64 |
| (with aluminum- | Long-term 3 months | 0.42 | 0.65 | 1104 | 751 | 68 |
| aluminum | Long-term 6 months | 0.43 | 0.67 | 1142 | 741 | 65 |
| packaging) | Long-term 12 months | 0.42 | 0.70 | 1131 | 708 | 63 |
| | Accelerated 1 month | 0.42 | 0.63 | 1194 | 776 | 65 |
| | Accelerated 3 months | 0.4 | 0.59 | 1103 | 717 | 65 |
| | Accelerated 6 months | 0.42 | 0.69 | 1205 | 746 | 62 |
| Example 32 | 0 month | 0.4 | 0.54 | 1492 | 940 | 63 |
| (with aluminum- | Long-term 3 months | 0.4 | 0.59 | 1490 | 954 | 64 |
| aluminum | Long-term 6 months | 0.41 | 0.58 | 1510 | 929 | 62 |
| packaging) | Long-term 12 months | 0.4 | 0.55 | 1465 | 920 | 63 |
| | Accelerated 1 month | 0.41 | 0.62 | 1505 | 978 | 65 |
| | Accelerated 3 months | 0.4 | 0.59 | 1476 | 930 | 63 |
| | Accelerated 6 months | 0.42 | 0.69 | 1526 | 931 | 61 |
| Example 33 | 0 month | 0.4 | 0.54 | 501 | 321 | 64 |
| (with aluminum- | Long-term 3 months | 0.37 | 0.61 | 500 | 320 | 64 |
| aluminum | Long-term 6 months | 0.38 | 0.70 | 530 | 331 | 62 |
| packaging) | Long-term 12 months | 0.38 | 0.77 | 522 | 318 | 61 |
| | Accelerated 1 month | 0.42 | 0.65 | 507 | 330 | 65 |
| | Accelerated 3 months | 0.4 | 0.53 | 499 | 319 | 64 |
| | Accelerated 6 months | 0.41 | 0.75 | 533 | 331 | 62 |

### Examples 34-39: Preparation of Formulations of Different Strengths

The preparation method comprises:
a. adding the prescribed amounts of Lactose 1, Lactose 2 and magnesium stearate to a high-shear mixer, thus obtaining an excipient mixture after mixing;
b. adding the excipient mixture and a prescribed amount of micronized compound of Formula (I) to a three-dimensional mixer, and passing them through a 60-mesh pharmacopoeia sieve after mixing with the excipient mixture to obtain a total mixed pharmaceutical powder;
b. filling 22 mg of the total mixed pharmaceutical powder into an empty hydroxypropyl methylcellulose capsule;
c. packaging the capsule with aluminum-aluminum packaging to obtain the formulations of Examples 34-39.

The following table shows the mass of each component per 100 g of the composition. Specifically, the Lactose 1 is Respitose® ML001, and the Lactose 2 is Lactohale® 300.

| | | Example | Example | Example | Example | Example | Example |
|---|---|---|---|---|---|---|---|
| Item | | 34 | 35 | 36 | 37 | 38 | 39 |
| Formulation strength (mg/capsule) | | 0.25 | 0.5 | 0.75 | 1 | 1.5 | 2 |
| Ingredient | Micronized compound of | 1.14 | 2.27 | 3.41 | 4.55 | 6.82 | 9.09 |
| | Formula (I) (g) | | | | | | |
| | Lactose 1 (g) | 94.80 | 93.71 | 92.62 | 91.53 | 89.35 | 87.17 |
| | Lactose 2 (g) | 3.95 | 3.90 | 3.86 | 3.81 | 3.72 | 3.63 |
| | Magnesium stearate (g) | 0.12 | 0.12 | 0.12 | 0.11 | 0.11 | 0.11 |
| Measurement | Mixing uniformity RSD (%) | 2 | 3 | 3 | 3 | 2 | 2 |
| | Amount delivered out of | 176 | 341 | 612 | 823 | 1101 | 1528 |
| | the inhaler device (μg) | | | | | | |
| | Fine particle dose FPD (μg) | 105 | 209 | 385 | 505 | 699 | 952 |
| | Fine particle fraction FPF | 60 | 61 | 63 | 61 | 63 | 62 |
| | (%) | | | | | | |

The compositions of the micronized compound of Formula (I), lactose and magnesium stearate exhibited good mixing uniformity, with RSD values of less than 5% in all cases. Compositions comprising different percentages of the micronized compound of Formula (I) all demonstrated high FPF (all greater than 60%).

### Examples 40-41: Compositions with Different Amounts of Diluent

### The Preparation Method for the Pharmaceutical Composition Comprises:

Weighing out the prescribed amount of lactose, the prescribed amount of magnesium stearate, and the prescribed amount of micronized compound of Formula (I); mixing them with a three-dimensional mixer to obtain a total mixed pharmaceutical powder;
The following table shows the mass of each component per 100 g of the composition.

| Item | | Example 40 | Example 41 |
|---|---|---|---|
| Ingredient | Micronized compound of Formula (I) (g) | 29.8 | 9.8 |
| | Lactose (g) | 70 | 90 |
| | Magnesium stearate (g) | 0.2 | 0.2 |
| Measurement | Bulk density (g/ml) | 0.565 | 0.644 |
| | Tapped density (g/ml) | 0.883 | 0.976 |
| | Compressibility index (%) | 36 | 34 |
| | Mixing uniformity (RSD, %) | 5 | 1 |

When the diluent was present at an above content, the composition exhibited good fluidity.

### Examples 42-43

The following preparation method was used:
a. first mixing step: weighing out one-twentieth of the prescribed amount of diluent, magnesium stearate, and the micronized powder of the compound of Formula (I), and mixing them with a three-dimensional mixer to obtain Mixture \textcircled{1};
b. second mixing step: adding the amount of Mixture \textcircled{1} and the remaining diluent to a high-shear mixer for mixing to obtain a total mixed pharmaceutical powder;
c. filling 23 mg of the total mixed pharmaceutical powder into a hydroxypropyl methylcellulose capsule;
d. packaging the capsule with aluminum-aluminum packaging to obtain the formulations of Examples 42-43.

| Ingredient | Example 42 | Example 43 |
|---|---|---|
| Micronized compound of Formula (I) (g) | 3.4 | 3.4 |
| Mannitol (g) | 92.62 | / |
| Leucine (g) | / | 92.62 |
| Lactose (Lactohale ®300, g) | 3.86 | 3.86 |
| Magnesium stearate (g) | 0.12 | 0.12 |

Examples 42-43: under the long-term condition of 30° C±2° C/65%+5% RH and the accelerated condition of 40° C±2° C/75%+5% RH, no significant increase in drug impurities or significant decrease in the fine particle fraction was observed. Consequently, the compositions obtained using different diluents exhibited high FPF values and good stability. The test results are shown in the following table:

| | | | | Amount | | |
|---|---|---|---|---|---|---|
| | | | | delivered | Fine | Fine |
| | | Related Substance (%) | | out of the | particle | particle |
| | | RRT = about | Total | inhaler | dose FPD | fraction |
| Investigation item | | 1.20 ≤ 1.0% | impurities ≤3.0% | device (μg) | (μg) | FPF (%) |
| Example | 0 month | 0.37 | 0.64 | 498 | 313 | 63 |
| 42 (with | Long-term 3 months | 0.36 | 0.69 | 532 | 345 | 65 |
| aluminum- | Accelerated 1 month | 0.38 | 0.61 | 508 | 325 | 64 |
| aluminum | Accelerated 3 months | 0.37 | 0.69 | 519 | 327 | 63 |
| packaging) | | | | | | |
| Example | 0 month | 0.34 | 0.60 | 516 | 320 | 62 |
| 43 (with | Long-term 3 months | 0.35 | 0.69 | 522 | 339 | 65 |
| aluminum- | Accelerated 1 month | 0.38 | 0.66 | 524 | 341 | 65 |
| aluminum | Accelerated 3 months | 0.37 | 0.63 | 513 | 323 | 63 |
| packaging) | | | | | | |

### Test Methods

### Related Substances and Methods:

To a 50 mL volumetric flask was added an appropriate amount of the powder sample and then an appropriate amount of the solvent water-acetonitrile-phosphoric acid (70:30:0.1). The mixture was subjected to ultrasonication to dissolve the compound of Formula (I), brought to room temperature, diluted to predetermined scale mark with the solvent, shaken well, and filtered to collect the subsequent filtrate after discarding an initial volume of the filtrate.

| Detection | | Column | Injection |
|---|---|---|---|
| Wavelength | Flow Rate | Temperature | Volume |
| 210 nm | 1.0 ml/min | 35 °C | 10 μl |

Mobile Phase A: [0.02 mol/L potassium dihydrogen phosphate solution (adjusted to pH 5.8 with sodium hydroxide test solution)]−acetonitrile (90:10)
Mobile Phase B: acetonitrile

Gradient elution was performed according to the following table:

| | Mobile Phase A | Mobile Phase B |
|---|---|---|
| Time (min) | (%) | (%) |
| 0 | 89 | 11 |
| 3 | 89 | 11 |
| 10 | 73 | 27 |
| 30 | 73 | 27 |
| 35 | 30 | 70 |
| 40 | 30 | 70 |
| 42 | 89 | 11 |
| 55 | 89 | 11 |

### Method for Fine Particle Dose:

It was determined through the Testing of Aerodynamic Properties of Fine Particles in Inhaled Formulations (Chinese Pharmacopoeia 2020 Edition, Volume IV, General Chapter 0951).

Solvent:ethanol-water-phosphoric acid (30:70:0.1).

Test solution: an appropriate amount of coating solution was added to each collection plate of the Next Generation Impactor (Chinese Pharmacopoeia 2020 Edition, Volume IV, General Chapter 0951, Device 3) and spread evenly. 15 mL of the solvent was added to the pre-separator. The inhaler device, adapter, Next Generation Impactor, and vacuum pump were connected, with the flow rate adjusted to 60±1 L per minute. The inhaler device was inserted into the adapter, and the vacuum pump was turned on for suction of approximately 4 seconds. The inhaler device, adapter and artificial throat, pre-separator, 1-7 stage collection plates, and MOC (a terminal Micro-Orifice Collector) stage collection plate were rinsed with the solvent. Dissolution was accomplished with the addition of the solvent. The above sample solutions were mixed well and filtered to collect the subsequent filtrate after discarding an initial volume of the filtrate.

Control solution: an appropriate amount of the compound of Formula (I) as a control was accurately weighed out, dissolved with the solvent and quantitatively diluted to prepare a solution containing approximately 30 μg per 1 ml.

The test solution and control solution were accurately measured out and injected into a liquid chromatograph. The chromatograms were recorded, and the amount of compound of Formula (I) in each test solution was quantified by the external standard method based on peak area. The fine particle dose featuring an aerodynamic diameter≤5 μm was calculated using the software.

| Detection | | Column | Injection |
|---|---|---|---|
| Wavelength | Flow Rate | Temperature | Volume |
| 366 nm | 1.0 ml/min | 30 °C | 10 μl |

Mobile phase: methanol-water-phosphoric acid (60:40:0.1)

### Method for Content:

To a 500 mL volumetric flask was added an appropriate amount of the powder and then an appropriate amount of a solvent to allow a 30-minute soak. The mixture was subjected to ultrasonication to dissolve the compound of Formula (I), brought to room temperature, diluted to predetermined scale mark with the solvent, shaken well, and filtered to collect the subsequent filtrate after discarding an initial volume of the filtrate.

| Detection | | Column | Injection |
|---|---|---|---|
| Wavelength | Flow Rate | Temperature | Volume |
| 336 nm | 1.0 ml/min | 30 °C | 20 μl |

Mobile Phase A: [0.02 mol/L potassium dihydrogen phosphate solution (adjusted to pH 5.8 with sodium hydroxide test solution)]−acetonitrile (90:10)
Mobile Phase B: acetonitrile

Gradient elution was performed according to the following table:

| | Mobile Phase A | Mobile Phase B |
|---|---|---|
| Time (min) | (%) | (%) |
| 0 | 76 | 24 |
| 3 | 76 | 24 |
| 4 | 72 | 28 |
| 15 | 70 | 30 |
| 18 | 70 | 30 |
| 20 | 40 | 60 |
| 22 | 40 | 60 |
| 24 | 76 | 24 |
| 30 | 76 | 24 |

### Method for Particle Size:

An appropriate amount of the powder was tested through the Particle Size and Particle Size Distribution Assay (Chinese Pharmacopoeia 2020 Edition, Volume IV, General Chapter 0982, Method 3). The Sympatec HELOS laser particle sizer with RODOS as the dispersion device was used for the assay according to the parameters in the following table, and characteristic values of the particle size distribution were recorded.

| Name | Parameter |
|---|---|
| Sample introduction method | Dry (RODOS) sample |
| | introduction |
| Optical lens | R1: 0.1/0.18-35 μm |
| Disperser type | RODOS/M |
| Sample introduction mode | Micromethod |
| Dispersion pressure | 3-5 bar |
| Test sample amount | Approximately 20-200 mg |

### Clinical Trial of the Compound of Formula (I) Dry Powder in COPD Subjects

A non-limiting example of a Multicenter, Single-Arm, Open-Label Study of the administration of the compound of formula (I) to COPD subjects with a Dry Powder Inhaler is described below.

Subjects aged 40 to 80 years with moderate-to-severe stable chronic obstructive pulmonary disease (COPD) are selected for the study based on the inclusion and exclusion criteria described below.

Dosage: 0.75 mg of the compound of formula (I) is administered twice a day with a Dry Powder Inhaler for 52 weeks; or 1.5 mg of the compound of formula (I) is administered twice a day with a Dry Powder Inhaler for 52 weeks; or 3 mg of the compound of formula (I) is administered twice a day with a Dry Powder Inhaler for 52 weeks.

Subjects are instructed to inhale the DPI formulation through the mouth twice a day. Key Inclusion Criteria:
1. Aged 40 to 80 years (inclusive) at the time of screening visit (Visit 1), male or female;
2. Subjects diagnosed with chronic obstructive pulmonary disease (COPD) in accordance with the GOLD 2026 diagnostic criteria prior to screening [GOLD 2026 criteria: presence of chronic respiratory symptoms such as dyspnea, chronic cough or sputum production, and/or a history of risk factor exposure, and the pulmonary function test results show: the forced expiratory volume in one second (FEV1) after using bronchodilators/forced vital capacity (FVC)<0.7];
3. At screening visit (Visit 1):
   Post-bronchodilator FEV1/FVC<0.7; and
   Post-bronchodilator FEV1≥30% and <80% of predicted value;
4. Modified Medical Research Council (mMRC) dyspnea scale score≥2 at screening.

### Key Exclusion Criteria:

1. Subjects with unresolved lower respiratory tract infection occurring within 6 weeks prior to screening (Visit 1) and/or before enrollment;
2. Severe or uncontrolled cardiovascular disease or history, including but not limited to: New York Heart Association (NYHA) functional class III/IV at screening; acute myocardial infarction, unstable angina, or acute coronary syndrome within 6 months prior to screening; clinically significant arrhythmia requiring intervention or marked QTcF prolongation (male>450 ms, female>470 ms) within 3 months prior to screening; or unstable or uncontrolled hypertension (systolic blood pressure≥160 mmHg or diastolic blood pressure≥100 mmHg) at screening;
3. History of malignancy (except for carcinoma in situ, cutaneous squamous cell carcinoma, and basal cell carcinoma cured for more than 5 years), suspected malignancy, or undetermined neoplasm;
4. Concurrent severe, uncontrolled renal, neurological, endocrine, thyroid, urological, ophthalmic, immunological, psychiatric, gastrointestinal, hepatic, or hematological diseases/abnormalities that, in the investigator's judgment, may pose a safety risk to the subject or confound study outcome analysis;
5. Known hypersensitivity to the compound of formula (I) inhalation powder, salbutamol, or any component of the drug delivery system;
6. Use of any protocol-prohibited medication prior to spirometry at screening (Visit 1);
7. History of lung lobectomy or lung volume reduction surgery within 12 months prior to screening;
8. Major surgery (requiring general anesthesia) within 6 weeks prior to screening with incomplete recovery, or planned surgery before study completion;
9. Subjects determined by the investigator to require oxygen therapy;
10. Clinically significant sleep apnea requiring continuous positive airway pressure (CPAP) or non-invasive positive pressure ventilation (NIPPV).

### Outcome Measurements:

The incidence of treatment-emergent adverse events (TEAEs), assessed through vital signs, physical examinations, laboratory tests, 12-lead ECG parameters, and detailed adverse event monitoring. The AFEV1 AUC0-12 h after the 4th week administration (the area under the curve representing the change in FEV1 from baseline to 12 hours after administration).

1-36. (canceled)

37. A dry powder pharmaceutical composition for inhalation with a dry powder inhaler (DPI), comprising:
a) a compound of Formula (I), or a pharmaceutically acceptable salt thereof; 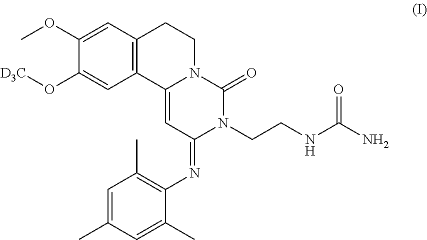 [SMILES: [2H]C([2H])([2H])Oc1cc2c(cc1OC)CCn1c-2c/c(=N\c2c(C)cc(C)cc2C)n(CCNC(N)=O)c1=O]
b) one or more diluents selected from lactose, mannitol, glycine, leucine, phospholipid, xylitol, and acacia gum; or
one or more lubricants selected from magnesium stearate, micronized silica gel, talcum powder, leucine, isoleucine, arginine, lysine, phenylalanine, tryptophan, methionine and polyethylene glycol; or
one or more diluents and one or more lubricants.

38. The pharmaceutical composition according to claim 37, wherein the composition comprises:
a) the compound of Formula (I), or a pharmaceutically acceptable salt thereof: 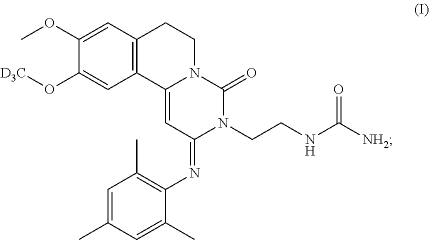 [SMILES: [2H]C([2H])([2H])Oc1cc2c(cc1OC)CCn1c-2c/c(=N\c2c(C)cc(C)cc2C)n(CCNC(N)=O)c1=O]
and
b) a diluent that is lactose and a lubricant that is magnesium stearate.

39. The pharmaceutical composition according to claim 38, wherein the composition comprises:
a) the compound of Formula (I): 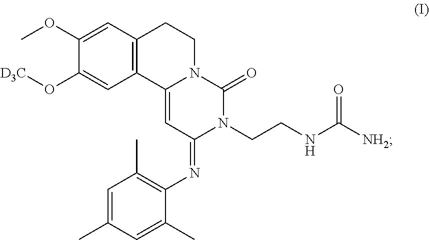 [SMILES: [2H]C([2H])([2H])Oc1cc2c(cc1OC)CCn1c-2c/c(=N\c2c(C)cc(C)cc2C)n(CCNC(N)=O)c1=O]
and
b) a diluent that is lactose and a lubricant that is magnesium stearate.

40. The pharmaceutical composition according to claim 39, wherein as calculated on the basis of the free form of the compound of Formula (I), the amount of the compound of Formula (I) is 0.5 g-12 g per 100 g of the pharmaceutical composition.

41. The pharmaceutical composition according to claim 39, wherein the diluent is lactose; and the amount of the diluent is 85 g-99.95 g per 100 g of the pharmaceutical composition.

42. The pharmaceutical composition according to claim 41, wherein the lactose consists of both coarse lactose and fine lactose having the following particle size ranges:
1. coarse lactose with a D90 of 100-220 μm; and
2. fine lactose with a D90 of less than 10 μm.

43. The pharmaceutical composition according to claim 42, wherein the weight percentage of the fine lactose relative to the total weight of the lactose is 2%-10%.

44. The pharmaceutical composition according to claim 37, wherein the pharmaceutical composition comprises one or more diluents and one or more lubricants,
the amount of the active ingredient is 0.5 g-12 g per 100 g of the pharmaceutical composition;
the amount of the one or more lubricants is 0.01 g-2 g per 100 g of the pharmaceutical composition; and
the amount of the one or more diluents is 85 g-99.5 g per 100 g of the pharmaceutical composition.

45. The pharmaceutical composition according to claim 39, wherein,
the weight ratio of the active ingredient to the lubricant is 1:0.002-1:0.5; and
the weight ratio of the active ingredient to the diluent is 1:7-1:50.

46. The pharmaceutical composition according to claim 45, wherein the particle size distribution of the compound of Formula (I) has a D50 value of 0.5 μm-5 μm and a D90 value of no greater than 10 μm.

47. The pharmaceutical composition according to claim 45, wherein the compound of Formula (I) comprises micronized compound of Formula (I) and the particle size distribution of the micronized compound of Formula (I) has a D50 value of 1 μm-3 μm and a D90 value of no greater than 5 μm.

48. The pharmaceutical composition according to claim 46, wherein the amount of the compound of Formula (I) in a unit dose is 0.1 mg-5 mg.

49. The pharmaceutical composition according to claim 46, wherein the amount of the compound of Formula (I) in a unit dose is 0.1 mg, 0.15 mg, 0.2 mg, 0.25 mg, 0.3 mg, 0.35 mg, 0.4 mg, 0.45 mg, 0.5 mg, 0.6 mg, 0.7 mg, 0.75 mg, 0.8 mg, 0.9 mg, 1 mg, 1.25 mg, 1.5 mg, 2 mg, 2.5 mg, 2.75 mg, 3 mg, or 4 mg.

50. The pharmaceutical composition according to claim 49, wherein the mass of the unit dose is about 5 mg to about 50 mg.

51. A method of treating an airway inflammatory disease in a human comprising administering to the human in need thereof one or two daily doses of the pharmaceutical composition of claim 46, wherein each dose is administered by inhalation with a dry powder inhaler (DPI);
wherein the airway inflammatory disease is chronic obstructive pulmonary disease (COPD), bronchiectasis, non-cystic fibrosis bronchiectasis, asthma, acute respiratory distress syndrome (ARDS), exercise-induced bronchoconstriction (EIB), chronic bronchitis, emphysema, alpha-1 antitrypsin deficiency, combined pulmonary fibrosis and emphysema (CPFE), pulmonary hypertension, cystic fibrosis, or asthma-COPD overlap syndrome (ACOS).

52. The method of claim 51, wherein each dose of the pharmaceutical composition comprises the compound of Formula (I), or a pharmaceutically acceptable salt thereof, in an amount equivalent to about 0.1 mg to about 6 mg of the compound of Formula (I).

53. The method of claim 51, wherein two daily doses of the pharmaceutical composition are administered, and each dose of the pharmaceutical composition comprises the compound of Formula (I), or a pharmaceutically acceptable salt thereof, in an amount equivalent to about 0.1 mg, about 0.15 mg, about 0.20 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, or about 4 mg of the compound of Formula (I).

54. The method of claim 51, wherein two daily doses of the pharmaceutical composition are administered, and each dose of the pharmaceutical composition comprises the compound of Formula (I), or a pharmaceutically acceptable salt thereof, in an amount equivalent to about 0.25 mg, about 0.5 mg, about 0.75 mg, about 1 mg, about 1.5 mg, about 2 mg, about 2.5 mg, about 3 mg, about 3.5 mg, or about 4 mg of the compound of Formula (I).

55. The method of claim 51, wherein the asthma is allergic asthma, aspirin-induced asthma, cough-variant asthma, exercise-induced asthma, nighttime asthma, steroid-resistant asthma, occupational asthma, eosinophilic asthma, neutrophilic asthma, or mixed eosinophilic and neutrophilic asthma.

56. The method of claim 51, wherein the airway inflammatory disease is chronic obstructive pulmonary disease (COPD) and treating COPD comprises lowering the annualized rate of pulmonary exacerbations, slowing the decline of lung function, prolonging the time to the first exacerbation, lowering the frequency of severe exacerbations, or combinations thereof, as compared to a human with COPD not being treated with the compound of Formula (I).
